(19) United States
(12) Patent Application Publication
(10) Pub. No.: US 2025/0325785 A1
(43) Pub. Date: Oct. 23, 2025

(54) Intermittent-Catheter Assemblies and Systems

(71) Applicant: C. R. Bard, Inc., Franklin Lakes, NJ (US)

(72) Inventors: Pooja Bhuvanesh Kulkarni, Pune (IN); Sudev Geetha Surendran, Kozhikode District (IN)

(21) Appl. No.: 18/641,181

(22) Filed: Apr. 19, 2024

(51) Int. Cl. A61M 25/00; A61F 5/451; B65D 85/14

(52) U.S. Cl. CPC A61M 25/0082; A61F 5/451; A61M 25/002; B65D 85/14; A61M 2202/0496

(57) ABSTRACT

Intermittent catheters in simple-to-use assemblies or systems thereof facilitate cleanliness before and after use. In an example, an intermittent-catheter system can include an intermittent catheter and packaging enclosing the intermittent catheter therein to maintain sterility of the intermittent catheter in an unused state of the intermittent-catheter system. The packaging can include a flexible packaging body and a packaging cover. The packaging body can be configured to hold a catheter tube of the intermittent catheter therein. The packaging cover can be configured to reveal a funnel of the intermittent catheter when an uncovering operation is performed. The uncovering operation can reveal an adhesive patch on either the packaging body or the packaging cover for adhering the packaging to a vertical surface for removal of the intermittent catheter from the packaging for catheterization. The packaging can be further configured for resealing the intermittent catheter therein subsequent to the catheterization.

### BACKGROUND

Users of urinary catheters such as intermittent catheters self-catheterize around four to six times a day on average. However, catheterization with such intermittent catheters can be messy. Indeed, existing intermittent catheters often include water or lubricant in packages thereof for lubrication of the intermittent catheters prior to insertion, but such packages are not configured to keep the water or lubricant from making messes. That, and such packages are not configured to keep post-catheterization messes from occurring such as those involving voided or residual urine in the intermittent catheters. What is needed are simple-to-use intermittent catheters that facilitate cleanliness before and after use.

Disclosed herein are intermittent-catheter assemblies and systems that address the foregoing.

### SUMMARY

Disclosed herein is an intermittent-catheter system including, in some embodiments, an intermittent-catheter assembly and a drainage bag. The intermittent-catheter assembly includes an intermittent catheter, a distal end piece, and a storage sheath. The intermittent catheter includes a catheter tube and a connecting funnel over a proximal end portion of the catheter tube for draining urine from the intermittent catheter. The distal end piece is slidably disposed over a distal end portion of the catheter tube. The storage sheath is between the connecting funnel and the distal end piece. A combination of the connecting funnel, the distal end piece, and the storage sheath is configured to enclose an entirety of the catheter tube therein. The drainage bag includes a connecting spout connected to the connecting funnel of the intermittent catheter in at least an unused state of the intermittent-catheter system.

In some embodiments, the connecting funnel of the intermittent catheter is connected to the connecting spout of the drainage bag by a hinge.

In some embodiments, the connecting funnel of the intermittent catheter includes a male fitting extending from the connecting funnel. The connecting spout of the drainage bag includes a female fitting within the connecting spout configured to receive the male fitting.

In some embodiments, the connecting funnel of the intermittent catheter is configured to drain the urine into the drainage bag through the connecting spout of the drainage bag when the hinge is in a closed state.

In some embodiments, the connecting spout of the drainage bag is configured to drain the urine from the drainage bag when the hinge is in an opened state.

In some embodiments, the distal end piece of the intermittent catheter includes a protective seal over a distal opening of the distal end piece in at least the unused state of the intermittent-catheter system. The protective seal is configured to maintain sterility of the catheter tube of the intermittent catheter within the combination of the connecting funnel, the distal end piece, and the storage sheath.

In some embodiments, the connecting funnel of the intermittent catheter is connected to the connecting spout of the drainage bag by a slip or lock fit.

In some embodiments, the connecting spout of the drainage bag includes a male fitting extending from the connecting spout. The connecting funnel of the intermittent catheter includes a female fitting within the connecting funnel configured to receive the male fitting.

In some embodiments, the connecting funnel of the intermittent catheter includes a fluid seal disposed within the female fitting of the connecting funnel. The fluid seal is configured to accept the male fitting of the connecting spout therethrough for draining the urine into the drainage bag through the connecting spout when the connecting funnel is connected to the connecting spout.

In some embodiments, the fluid seal is configured to close when the male fitting of the connecting spout is withdrawn from the female fitting of the connecting funnel, thereby preventing residual urine in the intermittent catheter from leaking from the intermittent catheter when the connecting funnel is disconnected from the connecting spout.

In some embodiments, the connecting spout of the drainage bag is configured to drain the urine from the drainage bag when the connecting funnel of the intermittent catheter is disconnected from the connecting spout.

In some embodiments, the male fitting of the connecting spout is configured to drain the urine from the drainage bag.

In some embodiments, the connecting spout of the drainage bag includes a side arm at an angle to the male fitting. The side arm is configured to drain the urine from the drainage bag.

In some embodiments, the connecting funnel is connected to the distal end piece of the intermittent-catheter assembly in an end-to-end connection through a first arm of the connecting funnel. The connecting funnel of the intermittent catheter is also connected to the connecting spout of the drainage bag through a second arm of the connecting funnel.

In some embodiments, the distal end piece of the intermittent-catheter assembly includes a first male fitting extending from the distal end piece. The first arm of the connecting funnel includes a first female fitting within the first arm configured to receive the first male fitting.

In some embodiments, the first arm of the connecting funnel includes a first fluid seal disposed within the first female fitting of the first arm. The first fluid seal is configured to accept the first male fitting of the distal end piece therethrough.

In some embodiments, the end-to-end connection between the first arm of the connecting funnel and the distal end piece of the intermittent-catheter assembly is configured to maintain sterility of the catheter tube of the intermittent catheter within the combination of the connecting funnel, the distal end piece, and the storage sheath in at least the unused state of the intermittent-catheter system.

In some embodiments, the end-to-end connection between the first arm of the connecting funnel and the distal end piece of the intermittent-catheter assembly is configured to prevent residual urine in the intermittent catheter from leaking from the intermittent catheter in a used state of the intermittent-catheter system.

In some embodiments, the first fluid seal disposed within the first female fitting of the first arm is configured to close when the first male fitting of the distal end piece is withdrawn from the first female fitting, thereby preventing the urine in the intermittent catheter from leaking from the first arm of the intermittent catheter while draining the urine into the drainage bag.

In some embodiments, the connecting spout of the drainage bag includes a second male fitting extending from the connecting spout. The second arm of the connecting funnel includes a second female fitting within the second arm configured to receive the second male fitting.

In some embodiments, the second arm of the connecting funnel includes a second fluid seal disposed within the second female fitting of the second arm. The second fluid seal is configured to accept the second male fitting of the connecting spout therethrough for draining the urine into the drainage bag through the connecting spout when the second arm of the connecting funnel is connected to the connecting spout.

In some embodiments, the second fluid seal disposed within the second female fitting of the second arm is configured to close when the second male fitting of the connecting spout is withdrawn from the second female fitting, thereby preventing residual urine in the intermittent catheter from leaking from the intermittent catheter when the connecting funnel is disconnected from the connecting spout.

In some embodiments, the drainage bag is in a compact rolled form in at least the unused state of the intermittent-catheter system.

In some embodiments, the drainage bag is in a compact bellowed form in at least the unused state of the intermittent-catheter system.

Also disclosed herein is an intermittent-catheter system including, in some embodiments, an intermittent-catheter assembly including a sealing member joining a distal end piece and a proximal end piece of the intermittent-catheter assembly together. The intermittent-catheter assembly includes an intermittent catheter including a catheter tube, the distal end piece slidably disposed over a distal end portion of the catheter tube, the proximal end piece fixedly disposed over a proximal end portion of the catheter tube for draining urine from the intermittent catheter, and a storage sheath between the distal end piece and the proximal end piece. A combination of the distal end piece, the proximal end piece, and the storage sheath is configured to enclose an entirety of the catheter tube therein. The sealing member is over both the distal end piece and the proximal end piece joining them together in at least an unused state of the intermittent-catheter system.

In some embodiments, the sealing member is over both a distal opening of the distal end piece and a proximal opening of the proximal end piece. The sealing member maintains sterility of the catheter tube of the intermittent catheter within the combination of the distal end piece, the proximal end piece, and the storage sheath.

In some embodiments, the sealing member is a sealing membrane. The sealing membrane includes an extension tab configured for grasping and peeling the sealing membrane away from the distal opening of the distal end piece and the proximal opening of the proximal end piece.

In some embodiments, the distal end piece includes an introducer membrane under the sealing membrane. The introducer membrane is configured to rupture when a catheter tip of the catheter tube is pushed through the introducer membrane.

In some embodiments, the distal end piece includes an introducer membrane under the sealing membrane. The introducer membrane includes one or more scores configured to allow a catheter tip of the catheter tube controllably therethrough when the catheter tip is gently pushed through the introducer membrane.

In some embodiments, the sealing member is a sealing cap. The sealing cap includes an extension tab configured for grasping and peeling a linear portion of the sealing cap from away from a remainder of the sealing cap, thereby disrupting a structural integrity of the sealing cap and loosening the sealing cap for removal from the intermittent-catheter assembly.

In some embodiments, the sealing member is a sealing cap. The sealing cap includes an extension tab configured for grasping and pulling a rip cord through the sealing cap, thereby disrupting a structural integrity of the sealing cap and loosening the sealing cap for removal from the intermittent-catheter assembly.

In some embodiments, the sealing member is over a distal opening of the distal end piece and around a proximal opening of the proximal end piece.

In some embodiments, the sealing member is a sealing cap. The sealing cap includes an extension tab configured for grasping and pulling a rip cord through the sealing cap, thereby disrupting a structural integrity of the sealing cap and loosening the sealing cap for removal from the intermittent-catheter assembly.

In some embodiments, the intermittent-catheter system further includes a drainage bag. The drainage bag includes a connecting spout connected to the proximal end piece of the intermittent-catheter assembly. The sealing cap and the drainage bag maintain sterility of the catheter tube of the intermittent catheter within the combination of the distal end piece, the proximal end piece, and the storage sheath in at least an unused state of the intermittent-catheter system.

Also disclosed herein is an intermittent-catheter system including, in some embodiments, an intermittent catheter and packaging enclosing at least the intermittent catheter therein to maintain sterility of the intermittent catheter in at least an unused state of the intermittent-catheter system. The intermittent catheter includes a catheter tube and a funnel disposed over a proximal end portion of the catheter tube for draining urine from the intermittent catheter. The packaging includes a flexible packaging body and a packaging cover. The packaging body is configured to hold at least the catheter tube of the intermittent catheter therein. The packaging cover is configured to reveal the funnel of the intermittent catheter when an uncovering operation is performed with the packaging cover. The uncovering operation reveals an adhesive patch on either the packaging body or the packaging cover for adhering the packaging to a vertical surface for removal of the intermittent catheter from the packaging for catheterization.

In some embodiments, the packaging cover is flexible with an anterior cover and a posterior cover. The anterior and posterior covers are coupled together through an extension tab at a tabbed end portion of each cover of the anterior and posterior covers. The anterior and posterior covers are also coupled together through an access end portion of the packaging body at a coupled end portion of each cover of the anterior and posterior covers.

In some embodiments, the anterior cover is folded over an opening in an anterior face of the packaging body. The posterior cover is folded over a posterior face of the packaging body. Both the anterior and posterior covers are configured to simultaneously peel away from the packaging body when the extension tab is pulled away from the access end portion of the packaging body in the uncovering operation.

In some embodiments, the packaging body includes a complementary extension tab to the extension tab of the packaging cover. The complementary extension tab is configured for grasping while pulling the extension tab of the packaging cover away from the access end portion of the packaging body in the uncovering operation.

In some embodiments, the adhesive patch is on an internal face of the posterior cover adhered to the packaging body prior to the uncovering operation. The adhesive patch is on an external face of the posterior cover subsequent to the uncovering operation for adhering the packaging to the vertical surface.

In some embodiments, an internal face of the anterior cover is adhered to the packaging body by an adhesive around either a perimeter of the internal face of the anterior cover or the opening in the anterior face of the packaging body. The adhesive is configured for resealing the intermittent catheter in the packaging subsequent to the catheterization for a storage state of the intermittent-catheter system.

In some embodiments, the adhesive is around the perimeter of the internal face of the anterior cover. A closed end portion of the packaging body opposite the access end portion thereof is adhered to the internal face of the anterior cover in at least the unused state of the intermittent-catheter system such that the packaging body is folded into a compact form.

In some embodiments, the closed end portion of the packaging body is released by the anterior cover when the extension tab of the packaging cover is pulled away from the access end portion of the packaging body in the uncovering operation.

In some embodiments, the packaging cover is a flexible wraparound cover including an anterior cover and a posterior cover having longitudinal end portions coupled together to form the wraparound cover.

In some embodiments, the anterior cover is over an opening in an anterior face of the packaging body. The posterior cover is over a posterior face of the packaging body. The anterior and posterior covers are configured to sequentially peel away from the packaging body when an extension tab extending from the anterior cover is pulled across an access end portion of the packaging body in the uncovering operation.

In some embodiments, the adhesive patch is on the posterior face of the packaging body adhered to an internal face of the posterior cover prior to the uncovering operation.

In some embodiments, an internal face of the anterior cover is adhered to the packaging body by an adhesive around the opening in the anterior face of the packaging body. The adhesive is configured for resealing the intermittent catheter in the packaging subsequent to the catheterization for a storage state of the intermittent-catheter system.

In some embodiments, the access end portion of the packaging body is configured to fold over a main portion of the packaging body for resealing the intermittent catheter in the packaging with the adhesive around the opening in the anterior face of the packaging body in the storage state of the intermittent-catheter system.

In some embodiments, an internal face of the anterior cover includes an additional adhesive patch of the packaging adjacent to the extension tab extending from the anterior cover. The additional adhesive patch is configured for securing the access end portion of the packaging body folded over the main portion of the packaging body with the wraparound cover when wrapped back around the packaging body in the used state of the intermittent-catheter system.

In some embodiments, the packaging cover is rigid with an anterior cover and a posterior tray configured to hold the funnel of the intermittent catheter. The anterior cover and the posterior tray are coupled together through a hinge at a hinged end portion of the packaging cover. The posterior tray is coupled to an access end portion of the packaging body at a coupled end portion of the packaging cover opposite the hinged end portion of the packaging cover.

In some embodiments, the anterior cover is snapped onto the posterior tray over an opening in the posterior tray. The anterior cover is configured to swing away from the opening in the posterior tray in accordance with the hinge when an extension tab extending from the anterior cover is pulled away from the access end portion of the packaging body in the uncovering operation.

In some embodiments, the adhesive patch is on an internal face of the anterior cover prior to the uncovering operation. The adhesive patch is on an external face of the posterior cover subsequent to the uncovering operation for adhering the packaging to the vertical surface.

In some embodiments, a closed end portion of the packaging body opposite the access end portion thereof is held between the anterior cover and the posterior tray in at least the unused state of the intermittent-catheter system such that the packaging body is folded into a compact form.

In some embodiments, the closed end portion of the packaging body is released by the packaging cover when the extension tab extending from the anterior cover is pulled away from the access end portion of the packaging body in the uncovering operation.

In some embodiments, the packaging cover is configured for resealing the intermittent catheter in the packaging subsequent to the catheterization by snapping the anterior cover back onto the posterior tray for a storage state of the intermittent-catheter system.

These and other features of the concepts provided herein will become more apparent to those of skill in the art in view of the accompanying drawings and following description, which describe particular embodiments of such concepts in greater detail.

### BRIEF DESCRIPTION OF DRAWINGS

FIG. 1 illustrates a first intermittent-catheter system including a first intermittent-catheter assembly and a first drainage bag in accordance with some embodiments.

FIG. 2 illustrates the first intermittent-catheter system having a first junction between the first intermittent-catheter assembly and the first drainage bag in accordance with some embodiments.

FIG. 3 illustrates the first intermittent-catheter system having a second junction between the first intermittent-catheter assembly and the first drainage bag in accordance with some embodiments.

FIG. 4 illustrates a ghosted view of the second junction between the first intermittent-catheter assembly and the first drainage bag in accordance with some embodiments.

FIG. 5 illustrates a second intermittent-catheter system including a second intermittent-catheter assembly and the first drainage bag in an unused state in accordance with some embodiments.

FIG. 6 illustrates the second intermittent-catheter system in an opened state in accordance with some embodiments.

FIG. 7 illustrates the second intermittent-catheter system in a used state in accordance with some embodiments.

FIG. 8 illustrates a detailed view of the first drainage bag in accordance with some embodiments.

FIG. 9 illustrates a second drainage bag in accordance with some embodiments.

FIG. 10 illustrates a third drainage bag in accordance with some embodiments.

FIG. 11 illustrates a third intermittent-catheter assembly having joined end pieces with a sealing membrane thereover in accordance with some embodiments.

FIG. 12 illustrates a detailed view of the joined end pieces of the third intermittent-catheter assembly in accordance with some embodiments.

FIG. 13 illustrates a ghosted view of a distal end piece of the end pieces of the third intermittent-catheter assembly in accordance with some embodiments.

FIG. 14 illustrates the third intermittent-catheter assembly having the joined end pieces with a first sealing cap thereover in accordance with some embodiments.

FIG. 15 illustrates the third intermittent-catheter assembly having the joined end pieces with a second sealing cap thereover in accordance with some embodiments.

FIG. 16 illustrates the third intermittent-catheter system including the third intermittent-catheter assembly having the joined end pieces with a third sealing cap thereover that accommodates the first drainage bag in accordance with some embodiments.

FIG. 17 illustrates a fourth intermittent-catheter system including a first intermittent catheter in a first packaging format transitioning from an unused state to an opened state in accordance with some embodiments.

FIG. 18 illustrates an anterior view of the fourth intermittent-catheter system in the opened state in accordance with some embodiments.

FIG. 19 illustrates a posterior view of the fourth intermittent-catheter system in the opened state in accordance with some embodiments.

FIG. 20 illustrates the fourth intermittent-catheter system including the first intermittent catheter in a modification of the first packaging format transitioning from an unused state to an opened state in accordance with some embodiments.

FIG. 21 illustrates an anterior view of the fourth intermittent-catheter system of FIG. 20 in the opened state in accordance with some embodiments.

FIG. 22 illustrates a posterior view of the fourth intermittent-catheter system of FIG. 20 in the opened state in accordance with some embodiments.

FIG. 23 illustrates a fifth intermittent-catheter system including a second packaging format in accordance with some embodiments.

FIG. 24 illustrates the fifth intermittent-catheter system transitioning from an unused state to an opened state in accordance with some embodiments.

FIG. 25 illustrates an anterior view of a sixth intermittent-catheter system including a third packaging format in an unused state in accordance with some embodiments.

FIG. 26 illustrates an anterior view of the sixth intermittent-catheter system in an opened state in accordance with some embodiments.

FIG. 27 illustrates a posterior view of the sixth intermittent-catheter system in the unused state in accordance with some embodiments.

FIG. 28 illustrates a posterior view of the sixth intermittent-catheter system in the opened state in accordance with some embodiments.

FIG. 29 illustrates the sixth intermittent-catheter system transitioning from the opened state to a storage state in accordance with some embodiments.

FIG. 30 illustrates an anterior view of a seventh intermittent-catheter system including some intermittent catheter in a fourth packaging format transitioning from an unused state to an opened state in accordance with some embodiments.

FIG. 31 illustrates a posterior view of the seventh intermittent-catheter system in the opened state in accordance with some embodiments.

FIG. 32 illustrates an anterior view of the seventh intermittent-catheter system transitioning from the opened state to a storage state in accordance with some embodiments.

FIG. 33 illustrates an eighth intermittent-catheter system including the first intermittent-catheter assembly in a fifth packaging format transitioning from an unused state to an opened state in accordance with some embodiments.

FIG. 34 illustrates the eighth intermittent-catheter system sitting on a horizontal surface in the opened state in accordance with some embodiments.

FIG. 35 illustrates the eighth intermittent-catheter system hanging from a vertical surface in the opened state in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

“Proximal” is used to indicate a portion, section, piece, element, or the like of a medical device intended to be near or relatively nearer to a clinician when the medical device is used on a patient. For example, a “proximal portion” or “proximal section” of the medical device includes a portion or section of the medical device intended to be near the clinician when the medical device is used on the patient. Likewise, a “proximal length” of the medical device includes a length of the medical device intended to be near the clinician when the medical device is used on the patient. A “proximal end” of the medical device is an end of the medical device intended to be near the clinician when the medical device is used on the patient. The proximal portion, the proximal section, or the proximal length of the medical device need not include the proximal end of the medical device. Indeed, the proximal portion, the proximal section, or the proximal length of the medical device can be short of the proximal end of the medical device. However, the proximal portion, the proximal section, or the proximal length of the medical device can include the proximal end of the medical device. Should context not suggest the proximal portion, the proximal section, or the proximal length of the medical device includes the proximal end of the medical device, or if it is deemed expedient in the following description, “proximal portion,” “proximal section,” or “proximal length” can be modified to indicate such a portion, section, or length includes an end portion, an end section, or an end length of the medical device for a “proximal end portion,” a “proximal end section,” or a “proximal end length” of the medical device, respectively.

“Distal” is used to indicate a portion, section, piece, clement, or the like of a medical device intended to be near, relatively nearer, or even in a patient when the medical device is used on the patient. For example, a “distal portion” or “distal section” of the medical device includes a portion or section of the medical device intended to be near, relatively nearer, or even in the patient when the medical device is used on the patient. Likewise, a “distal length” of the medical device includes a length of the medical device intended to be near, relatively nearer, or even in the patient when the medical device is used on the patient. A “distal end” of the medical device is an end of the medical device intended to be near, relatively nearer, or even in the patient when the medical device is used on the patient. The distal portion, the distal section, or the distal length of the medical device need not include the distal end of the medical device. Indeed, the distal portion, the distal section, or the distal length of the medical device can be short of the distal end of the medical device. However, the distal portion, the distal section, or the distal length of the medical device can include the distal end of the medical device. Should context not suggest the distal portion, the distal section, or the distal length of the medical device includes the distal end of the medical device, or if it is deemed expedient in the following description, “distal portion,” “distal section,” or “distal length” can be modified to indicate such a portion, section, or length includes an end portion, an end section, or an end length of the medical device for a “distal end portion,” a “distal end section,” or a “distal end length” of the medical device, respectively.

An unused state of an intermittent-catheter assembly, system, or any component thereof indicates the intermittent-catheter assembly, system, or component thereof being described remains intact as manufactured, distributed, sold, or the like.

A used state of an intermittent-catheter assembly, system, or any component thereof indicates the intermittent-catheter assembly, system, or component thereof being described is not in an unused state; that is, the intermittent-catheter assembly, system, or component thereof is opened, unpackaged, prepared for use in a catheterization procedure, used in such a catheterization procedure, or the like.

A storage state of an intermittent-catheter assembly, system, or any component thereof indicates the intermittent-catheter assembly, system, or component thereof being described has been opened, unpackaged, prepared for use in a catheterization procedure, used in such a catheterization procedure, or the like but repackaged in some manner for storage until disposal is possible. Notably, packaging for intermittent-catheter systems set forth herein is configured to provide leak-free storage of one or more components of the intermittent-catheter systems after catheterization until disposal is possible. Alternatively, the storage state of an intermittent-catheter assembly, system, or any component thereof can indicate the intermittent-catheter assembly, system, or component thereof being described remains packaged as manufactured, distributed, sold, or the like in accordance with the unused state. Therefore, context should be used to inform any interpretation of the storage state of the intermittent-catheter assembly, system, or component thereof.

A closed state of an intermittent-catheter assembly, system, or any component thereof indicates the intermittent-catheter assembly, system, or component thereof being described is simply closed. As such, the intermittent-catheter assembly, system, or component thereof being described can be in an unused state, a used state (but closed), or a storage state. Therefore, context should be used to inform any interpretation of the closed state of the intermittent-catheter assembly, system, or component thereof beyond simply being closed.

An opened state of an intermittent-catheter assembly, system, or component thereof indicates the intermittent-catheter assembly, system, or component thereof being described is simply opened. As such, the intermittent-catheter assembly, system, or component thereof being described is also in a used state, as above. Indeed, whether or not the intermittent-catheter assembly, system, or any component thereof has been prepared for use in a catheterization procedure or used in such a catheterization procedure, sterility of the intermittent-catheter assembly, system, or component thereof is potentially compromised.

Unless defined otherwise, all technical and scientific terms used herein have the same meaning as commonly understood by those of ordinary skill in the art.

As set forth above, users of urinary catheters such as intermittent catheters self-catheterize around four to six times a day on average. However, catheterization with such intermittent catheters can be messy. Indeed, existing intermittent catheters often include water or lubricant in packages thereof for lubrication of the intermittent catheters prior to insertion, but such packages are not configured to keep the water or lubricant from making messes. That, and such packages are not configured to keep post-catheterization messes from occurring such as those involving voided or residual urine in the intermittent catheters. What is needed are simple-to-use intermittent catheters that facilitate cleanliness before and after use.

Disclosed herein are intermittent-catheter assemblies and systems that address the foregoing.

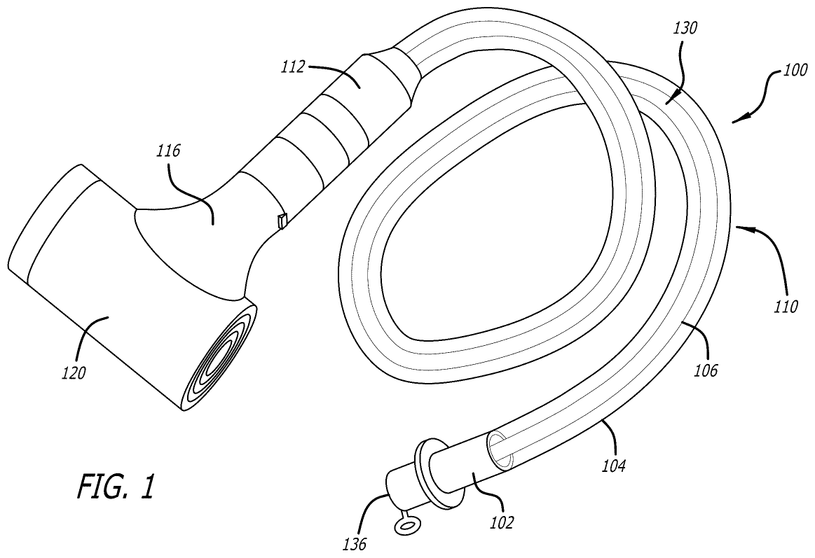

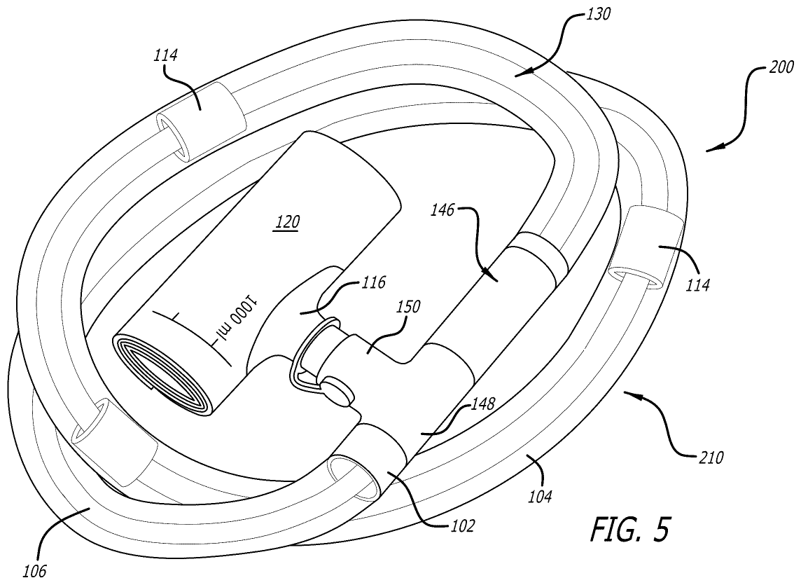

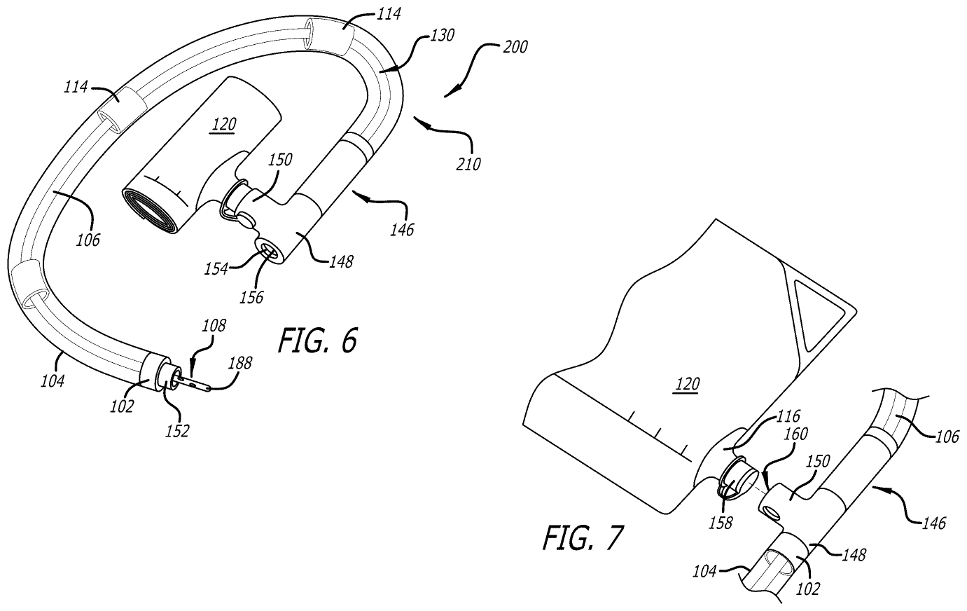

FIGS. 1-10 illustrate intermittent-catheter systems 100 and 200 in accordance with some embodiments. Indeed, FIGS. 1-4 illustrate the intermittent-catheter system 100 including an intermittent-catheter assembly 110 and a drainage bag 120 in accordance with some embodiments. Notably, the intermittent-catheter system 100 can have different junctions between the intermittent-catheter assembly 110 and the drainage bag 120 as shown among FIGS. 1-4, and the intermittent-catheter system 100 can have different drainage bags as shown among FIGS. 1 and 8-10. In addition, FIGS. 5-7 illustrate the intermittent-catheter system 200 including an intermittent-catheter assembly 210 and the drainage bag 120 in accordance with some embodiments. Notably, the intermittent-catheter system 200 can also have different drainage bags as shown among FIGS. 5-7 and 8-10.

As shown, the intermittent-catheter system 100 or 200 includes the intermittent-catheter assembly 110 or 210 and the drainage bag 120. While not shown, it should be understood that the intermittent-catheter system 100 or 200 can include additional catheterization components such as gloves, an antiseptic, an underpad, instructions for use, or some combination thereof.

The intermittent-catheter assembly 110 or 210 includes an intermittent catheter 130, a distal end piece 102, and a storage sheath 104. Notably, being that the intermittent-catheter assembly 110 can have the drainage bag 120 fixedly attached thereto, as shown, the intermittent-catheter assembly 110 can be synonymous with the intermittent-catheter system 100 in some embodiments notwithstanding any additional catheterization components.

The intermittent catheter 130 includes a catheter tube 106, a plurality of eyelets 108 in a distal end portion of the catheter tube 106, and a connecting funnel 112 fixedly disposed over a proximal end portion of the catheter tube 106 for draining urine from the intermittent catheter 130. The intermittent catheter 130 can be configured for use by any sex (e.g., male or female) or all sexes (i.e., unisex).

A protection mechanism including a combination of the connecting funnel 112 of the intermittent catheter 130, the distal end piece 102 of the intermittent-catheter assembly 110 or 210, and the storage sheath 104 of the intermittent-catheter assembly 110 or 210 between the connecting funnel 112 and the distal end piece 102 is configured to enclose an entirety of the catheter tube 106 of the intermittent catheter 130 therein. Such a protection mechanism is configured to maintain sterility of the catheter tube 106 of the intermittent catheter 130 in an unused state of the intermittent-catheter system 100 or 200 or the intermittent-catheter assembly 110 or 210 thereof. When lubricated with either a wet or dry lubricant, the protection mechanism is also configured to maintain lubricity of the catheter tube 106 in the unused state of the intermittent-catheter system 100 or 200 or the intermittent-catheter assembly 110 or 210 thereof. Lastly, the protection mechanism is also configured to prevent residual urine from leaking from the intermittent-catheter assembly 110 or 210 in a storage state of the intermittent-catheter system 100 or 200 or the intermittent-catheter assembly 110 or 210 thereof.

While the connecting funnel 112 of the intermittent catheter 130 is fixedly disposed over the proximal end portion of the catheter tube 106 of the intermittent catheter 130, the distal end piece 102 of the intermittent-catheter assembly 110 or 210 is slidably disposed over a distal end portion of the catheter tube 106 in the storage state of the intermittent-catheter system 100 or 200 or the intermittent-catheter assembly 110 or 210 thereof. However, in an opened state of the intermittent-catheter system 100 or 200 or the intermittent-catheter assembly 110 or 210 thereof such as when the intermittent-catheter assembly 110 or 210 is prepared for catheterization, during the catheterization, or subsequent to the catheterization, the distal end piece 102 is slidably disposed over a medial portion of the catheter tube 106 or the distal end portion of the catheter tube 106 with the storage sheath 104 collapsed between the distal end piece 102 and the connecting funnel 112 of the intermittent catheter 130.

The storage sheath 104 of the intermittent-catheter assembly 110 or 210 is a thin-walled (e.g., 25-125 μm), relatively small-diameter tube between the connecting funnel 112 and the distal end piece 102 configured to enclose an entirety of the catheter tube 106 of the intermittent catheter 130 therein. Such a storage sheath 104 can be formed of any polymer selected from at least a thermoplastic polyurethane, a thermoplastic elastomer, a polyamide such as a nylon or Pebax®, a thermoplastic polyester such as Hytrel® or Arnitel®, an ethylene vinyl acetate, a polyvinylidene fluoride, a polypropylene, a polyethylene, and the like.

Notably, the intermittent-catheter assembly 110 or 210 can further include one or more annular members 114 slidably disposed over the catheter tube 106 of the intermittent catheter 130 but under the storage sheath 104 between the connecting funnel 112 of the intermittent catheter 130 and the distal end piece 102 of the intermittent-catheter assembly 110 or 210. Being as the catheter tube 106 of the intermittent catheter 130 can be quite slender, the one-or-more annular members 114 are configured as stocky handling points for low-dexterity users when handling the intermittent-catheter assembly 110 or 210 during catheterization, particularly, catheter insertion. Advantageously, when the distal end piece 102 of the intermittent-catheter assembly 110 or 210 is slidably disposed over the medial portion of the catheter tube 106 or the distal end portion of the catheter tube 106 as in the foregoing opened state of the intermittent-catheter system 100 or 200 or the intermittent-catheter assembly 110 or 210 thereof, the distal end piece 102 can be combined with a number of the one-or-more annular members 114 proximal to the distal end piece 102 into which the distal end piece 102 slid when transitioning from the unused state to the opened state of the intermittent-catheter system 100 or 200 or the intermittent-catheter assembly 110 or 210 thereof. Such a combination of the distal end piece 102 and the number of the one-or-more annular members 114 forms a conglomerated handle for low-dexterity users when handling the intermittent-catheter assembly 110 or 210 during the catheterization, particularly, the catheter insertion.

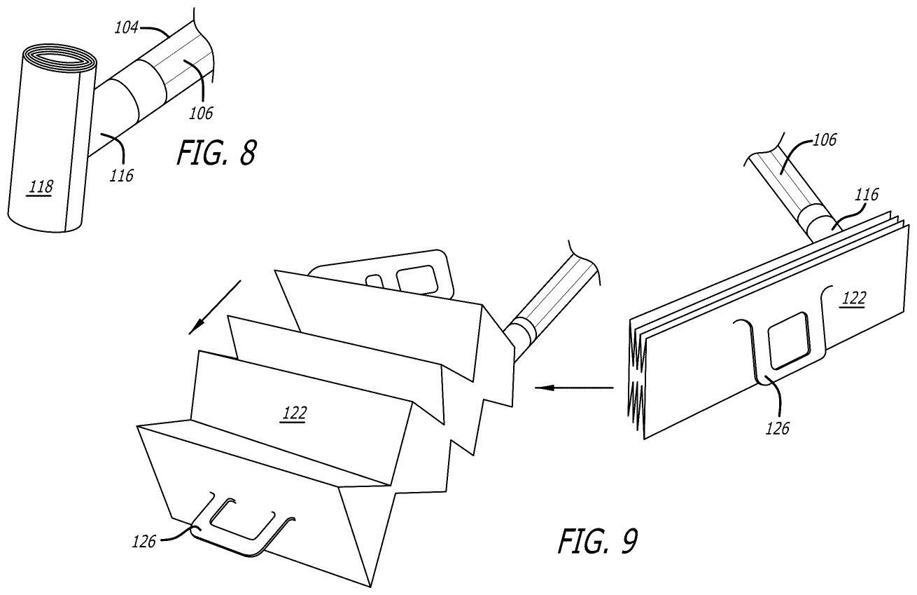

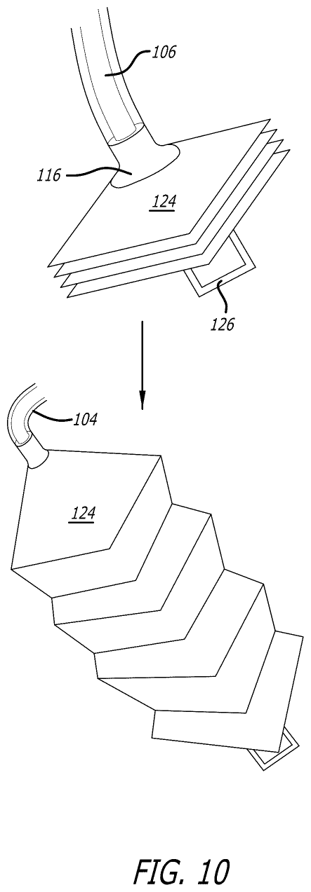

The drainage bag 120 includes a connecting spout 116 connected to the connecting funnel 112 of the intermittent catheter 130 in at least the unused state of the intermittent-catheter system 100 or 200. Such a drainage bag 120 can be in a compact form in the intermittent-catheter system 100 or 200, wherein the compact form is selected from at least a rolled form and a bellowed form. Indeed, FIG. 8 illustrates a rolled drainage bag 118, and FIGS. 9 and 10 illustrate bellowed drainage bags 122 and 124. Notably, each drainage bag of the bellowed drainage bags 122 and 124 has a different transverse cross section, specifically a rectangular cross section for the bellowed drainage bag 122 and a square cross section for the bellowed drainage bag 124, choice of which depends upon packaging for the intermittent-catheter system 100 or 200, some of which is set forth below. Regardless, each drainage bag of the bellowed drainage bags 122 and 124 can include a pull handle 126 opposite the connecting spout 116 thereof for grasping and expanding the bellowed drainage bag 122 or 124 therewith. Such a pull handle 126 can be out of line or off set from the connecting spout 116 as shown in FIG. 9 for the bellowed drainage bag 122; however, it can be advantageous for low-dexterity users to have the pull handle 126 in line with the connecting spout 116 as shown in FIG. 10 for the bellowed drainage bag 124 for even application of opposing tensile forces when expanding the bellowed drainage bag 122.

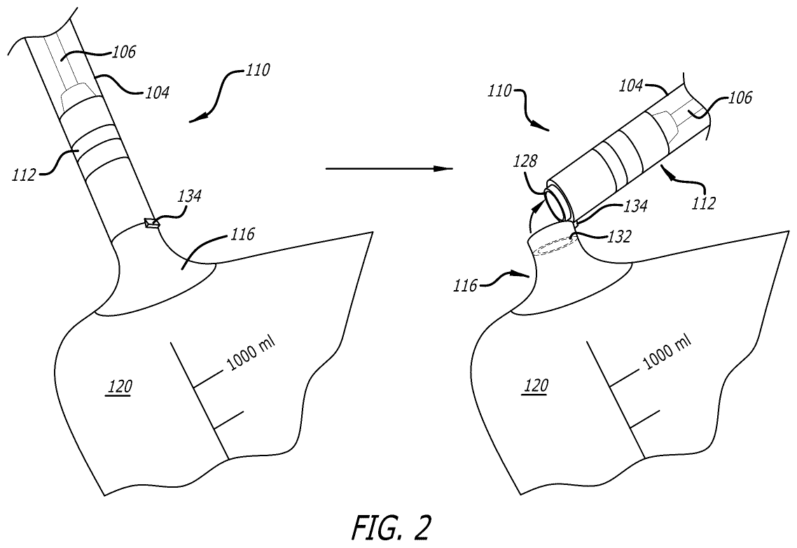

Adverting to the intermittent-catheter system 100 and some particular features thereof, FIGS. 1 and 2 illustrate the intermittent-catheter system 100 including a hinged junction between the intermittent-catheter assembly 110 and the drainage bag 120 in accordance with some embodiments.

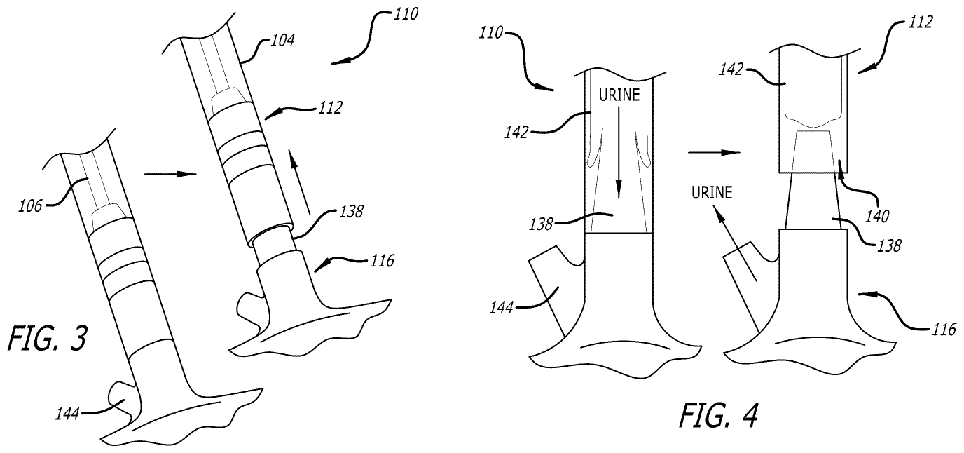

As shown, the connecting funnel 112 of the intermittent catheter 130 includes a male fitting 128 extending from the connecting funnel 112, and the connecting spout 116 of the drainage bag 120 includes a female fitting 132 within the connecting spout 116 configured to receive the male fitting 128 in the hinged junction. However, the inverse of such a fitting configuration in the hinged junction is possible as shown in FIGS. 3 and 4. Indeed, in the inverse fitting configuration, the connecting spout 116 of the drainage bag 120 includes the male fitting 128 extending from the connecting spout 116, and the connecting funnel 112 of the intermittent catheter 130 includes the female fitting 132 within the connecting funnel 112 configured to receive the male fitting 128. Regardless of the fitting configuration, which is not limited to the foregoing fitting configurations, the connecting funnel 112 of the intermittent catheter 130 is connected to the connecting spout 116 of the drainage bag 120 by a hinge 134 such as a living hinge between the connecting funnel 112 and the connecting spout 116.

The connecting funnel 112 of the intermittent catheter 130 is configured to drain urine into the drainage bag 120 through the connecting spout 116 of the drainage bag 120 when the hinged junction or the hinge 134 thereof is in a closed state. In such a closed state, central axes of the connecting funnel 112 of the intermittent catheter 130 and the connecting spout 116 of the drainage bag 120 are aligned with each other. While not shown, it should be understood the connecting funnel 112 of the intermittent catheter 130 and the connecting spout 116 of the drainage bag 120 can be optionally fastened together in the closed state of the hinged junction or the hinge 134 thereof by a clasp opposite the hinge 134. The connecting spout 116 of the drainage bag 120 is configured to drain urine from the drainage bag 120 when the hinged junction or the hinge 134 thereof is in an opened state. In such an opened state, the central axes of the connecting funnel 112 of the intermittent catheter 130 and the connecting spout 116 of the drainage bag 120 are not aligned with each other. For example, the central axes of the connecting funnel 112 of the intermittent catheter 130 and the connecting spout 116 of the drainage bag 120 can be at an acute, right, or obtuse angle to each other in the opened state of the hinged junction or the hinge 134 thereof.

Continuing with the intermittent-catheter system 100 of FIGS. 1 and 2, the intermittent-catheter system 100 includes a protective seal 136 as part of the protection mechanism in accordance with some embodiments.

As shown, the distal end piece 102 of the intermittent catheter 130 includes the protective seal 136 over a distal opening of the distal end piece 102 in at least the unused state of the intermittent-catheter system 100. The protective seal 136 is further configured to maintain the sterility of the catheter tube 106 of the intermittent catheter 130 within the combination of the connecting funnel 112, the distal end piece 102, and the storage sheath 104.

Continuing with the intermittent-catheter system 100 and some particular features thereof, FIGS. 3 and 4 illustrate the intermittent-catheter system 100 alternatively including a slip-fitted junction between the intermittent-catheter assembly 110 and the drainage bag 120 in accordance with some embodiments.

As shown, the connecting spout 116 of the drainage bag 120 includes a male fitting 138 extending from the connecting spout 116, and the connecting funnel 112 of the intermittent catheter 130 includes a female fitting 140 within the connecting funnel 112 configured to receive the male fitting 138 in the slip-fitted junction. However, the inverse of such a fitting configuration in the slip-fitted junction is possible as shown in FIG. 2. Indeed, in the inverse fitting configuration, the connecting funnel 112 of the intermittent catheter 130 includes the male fitting 138 extending from the connecting funnel 112, and the connecting spout 116 of the drainage bag 120 includes the female fitting 140 within the connecting spout 116 configured to receive the male fitting 138. Regardless of the fitting configuration, which is not limited to the foregoing fitting configurations, the connecting spout 116 of the drainage bag 120 is connected to the connecting funnel 112 of the intermittent catheter 130 by a slip fit such as that between the connecting spout 116 and the connecting funnel 112. That said, the slip fit can include a locking mechanism between the connecting spout 116 of the drainage bag 120 and the connecting funnel 112 of the intermittent catheter 130 such as complementary threads, thereby providing a lock fit for a lock-fitted junction between the connecting spout 116 and the connecting funnel 112.

The connecting funnel 112 of the intermittent catheter 130 is configured to drain urine into the drainage bag 120 through the connecting spout 116 of the drainage bag 120 when the connecting funnel 112 is connected to the connecting spout 116. Indeed, the connecting funnel 112 of the intermittent catheter 130 includes a fluid seal 142 disposed within the female fitting 140 of the connecting funnel 112. The fluid seal 142 is configured to accept therethrough the male fitting 138 of the connecting spout 116 of the drainage bag 120 for draining urine into the drainage bag 120 through the connecting funnel 112 of the intermittent catheter 130 when the connecting funnel 112 is connected to the connecting spout 116. The fluid seal 142 is configured to close when the male fitting 138 of the connecting spout 116 of the drainage bag 120 is withdrawn from the female fitting 140 of the connecting funnel 112 of the intermittent catheter 130, thereby preventing residual urine in the intermittent catheter 130 from leaking from the intermittent catheter 130 when the connecting funnel 112 is disconnected from the connecting spout 116.

The connecting spout 116 of the drainage bag 120 is configured to drain urine from the drainage bag 120 when the connecting funnel 112 of the intermittent catheter 130 is disconnected from the connecting spout 116. Indeed, the male fitting 138 of the connecting spout 116 of the drainage is configured to drain urine from the drainage bag 120. Alternatively, the connecting spout 116 of the drainage bag 120 includes a side arm 144 at an angle to the male fitting 138. The side arm 144 of the connecting spout 116 is configured to drain urine from the drainage bag 120 when the connecting funnel 112 of the intermittent catheter 130 is connected to the connecting spout 116 of the drainage bag 120 or disconnected from the connecting spout 116.

Adverting to the intermittent-catheter system 200 and some particular features thereof, FIGS. 5-7 illustrate the intermittent-catheter system 200 in which the connecting funnel 112 of the intermittent catheter 130 is branched for a branched connecting funnel 146 in accordance with some embodiments.

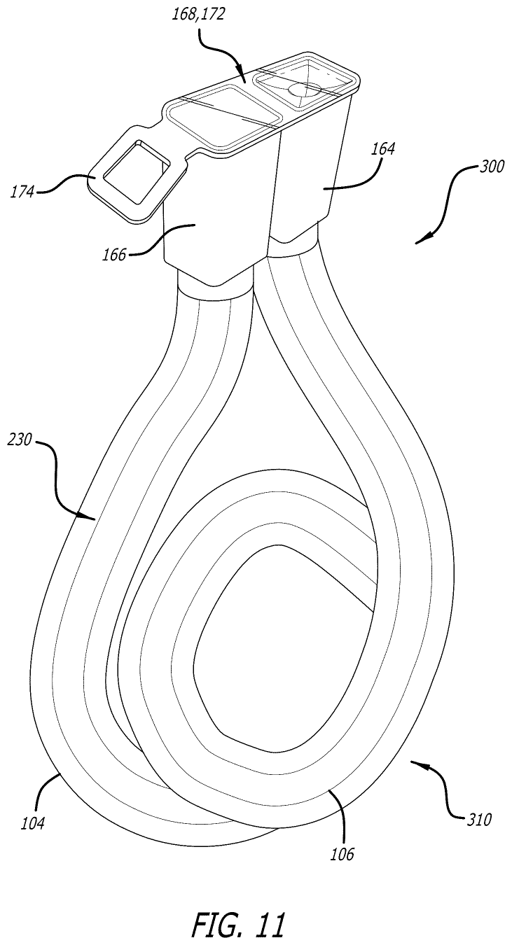

As shown, the connecting funnel 146 of the intermittent catheter 130 of the intermittent-catheter system 200 includes a first arm 148 of the connecting funnel 146 and a second arm 150 of the connecting funnel 146. The connecting funnel 146 of the intermittent catheter 130 so branched allows the intermittent-catheter system 200 or the intermittent-catheter assembly 210 thereof to be bent to form an end-to-end connection akin to that of a serpent eating its own tail (e.g., the ouroboros of ancient Egyptian iconography), thereby providing a compact form thereof. Indeed, the connecting funnel 146 of the intermittent catheter 130 is connected to the distal end piece 102 of the intermittent-catheter assembly 210 in such an end-to-end connection through the first arm 148 of the connecting funnel 146. Concomitantly, the connecting funnel 146 of the intermittent catheter 130 is also connected to the connecting spout 116 of the drainage bag 120 through the second arm 150 of the connecting funnel 146. Notably, FIGS. 11-16 illustrate the intermittent-catheter system 300 or the intermittent-catheter assembly 310 thereof in an alternative end-to-end compact form thereof.

The distal end piece 102 of the intermittent-catheter assembly 210 includes a first male fitting 152 extending from the distal end piece 102, and the first arm 148 of the connecting funnel 146 of the intermittent catheter 130 includes a first female fitting 154 within the first arm 148 of the connecting funnel 146 configured to receive the first male fitting 152 of the distal end piece 102. However, the inverse of such a fitting configuration in the end-to-end connection is possible, where the first arm 148 of the connecting funnel 146 of the intermittent catheter 130 includes the first male fitting 152 extending from the connecting funnel 146, and the distal end piece 102 of the intermittent-catheter assembly 210 includes the first female fitting 154 within the distal end piece 102 configured to receive the first male fitting 152 of the connecting funnel 146. Regardless of the fitting configuration, which is not limited to the foregoing fitting configurations, the end-to-end connection of the distal end piece 102 of the intermittent-catheter assembly 210 and the first arm 148 of the connecting funnel 146 of the intermittent catheter 130 effectively provides a protective seal over a distal opening of the distal end piece 102 in both the unused and storage states of the intermittent-catheter system 200 or the intermittent-catheter assembly 210 thereof.

When the distal end piece 102 of the intermittent-catheter assembly 210 includes the first male fitting 152, and when the first arm 148 of the connecting funnel 146 of the intermittent catheter 130 correspondingly includes the first female fitting 154, the first arm 148 of the connecting funnel 146 includes a first fluid seal 156 disposed within the first female fitting 154 of the first arm 148 of the connecting funnel 146 configured to accept the first male fitting 152 of the distal end piece 102 therethrough, thereby opening the first arm 148 of the connecting funnel 146. (See e.g., FIG. 4, which illustrates the fluid seal 142 disposed within the female fitting 140 of the connecting funnel 112 configured to accept therethrough the male fitting 138 of the connecting spout 116 of the drainage bag 120.) Alternatively, the first fluid seal 156 is absent from the first female fitting 154 of the first arm 148 of the connecting funnel 146 of the intermittent catheter 130. Regardless of the presence or absence of the first fluid seal 156, the end-to-end connection between the first arm 148 of the connecting funnel 146 of the intermittent catheter 130 and the distal end piece 102 of the intermittent-catheter assembly 210 is configured to maintain sterility of the catheter tube 106 of the intermittent catheter 130 within the combination of the connecting funnel 146, the distal end piece 102, and the storage sheath 104 in the unused state of the intermittent-catheter system 200 or the intermittent-catheter assembly 210 thereof. Further, the end-to-end connection between the first arm 148 of the connecting funnel 146 of the intermittent catheter 130 and the distal end piece 102 of the intermittent-catheter assembly 210 is configured to prevent residual urine in the intermittent catheter 130 from leaking from the intermittent catheter 130 in the used state of the intermittent-catheter system 200 or the intermittent-catheter assembly 210 thereof.

Further, the first fluid seal 156 disposed within the first female fitting 154 of the first arm 148 of the connecting funnel 146 of the intermittent catheter 130 is configured to close when the first male fitting 152 of the distal end piece 102 of the intermittent-catheter assembly 210 is withdrawn from the first female fitting 154 of the first arm 148 of the connecting funnel 146, thereby closing the first arm 148 of the connecting funnel 146 and preventing urine in the intermittent catheter 130 from leaking from the first arm 148 of the connecting funnel 146 while draining urine into the drainage bag 120. As set forth above, the first fluid seal 156 can be absent from the first female fitting 154 of the first arm 148 of the connecting funnel 146 of the intermittent catheter 130. In such embodiments, the intermittent-catheter system 200 benefits from a first end cap (not shown) or some other capping means for capping the first arm 148 of the connecting funnel 146 of the intermittent catheter 130 while draining urine into the drainage bag 120 through the second arm 150 of the connecting funnel 146 of the intermittent catheter 130 when the connecting funnel 146 is connected to the connecting spout 116 of the drainage bag 120 thereby.

The connecting spout 116 of the drainage bag 120 includes a second male fitting 158 extending from the connecting spout 116, and the second arm 150 of the connecting funnel 146 of the intermittent catheter 130 includes a second female fitting 160 within the second arm 150 of the connecting funnel 146 configured to receive the second male fitting 158 of the connecting spout 116. However, the inverse of such a fitting configuration is possible, where the second arm 150 of the connecting funnel 146 of the intermittent catheter 130 includes the second male fitting 158 extending from the connecting funnel 146, and the connecting spout 116 of the drainage bag 120 includes the second female fitting 160 within the connecting spout 116 configured to receive the second male fitting 158 of the connecting funnel 146. Regardless of the fitting configuration, which is not limited to the foregoing fitting configurations, connection of the second arm 150 of the connecting funnel 146 of the intermittent catheter 130 and the connecting spout 116 of the drainage bag 120 is configured for draining urine into the drainage bag 120 through the connecting spout 116.

When the connecting spout 116 of the drainage bag 120 includes the second male fitting 158, and when the second arm 150 of the connecting funnel 146 of the intermittent catheter 130 correspondingly includes the second female fitting 160, the second arm 150 of the connecting funnel 146 includes a second fluid seal (not shown) disposed within the second female fitting 160 of the second arm 150 of the connecting funnel 146 configured to accept the second male fitting 158 of the connecting spout 116 therethrough, thereby opening the second arm 150 of the connecting funnel 146. (See e.g., FIG. 4, which illustrates the fluid seal 142 disposed within the female fitting 140 of the connecting funnel 112 configured to accept therethrough the male fitting 138 of the connecting spout 116 of the drainage bag 120.) Alternatively, the second fluid seal is absent from the second female fitting 160 of the second arm 150 of the connecting funnel 146 of the intermittent catheter 130. Regardless of the presence or absence of the second fluid seal, the connection of the second arm 150 of the connecting funnel 146 of the intermittent catheter 130 and the connecting spout 116 of the drainage bag 120 is configured for draining urine into the drainage bag 120 through the connecting spout 116.

Further, the second fluid seal disposed within the second female fitting 160 of the second arm 150 of the connecting funnel 146 of the intermittent catheter 130 is configured to close when the second male fitting 158 of the connecting spout 116 of the drainage bag 120 is withdrawn from the second female fitting 160 of the second arm 150 of the connecting funnel 146, thereby closing the second arm 150 of the connecting funnel 146 and preventing residual urine in the intermittent catheter 130 from leaking from the second arm 150 of the connecting funnel 146 when the connecting funnel 146 is disconnected from the connecting spout 116. As set forth above, the second fluid seal can be absent from the second female fitting 160 of the second arm 150 of the connecting funnel 146 of the intermittent catheter 130. In such embodiments, the intermittent-catheter system 200 benefits from a second end cap (not shown) or some other capping means for capping the second arm 150 of the connecting funnel 146 of the intermittent catheter 130 when the connecting funnel 146 is disconnected from the connecting spout 116 of the drainage bag 120.

FIGS. 11-16 illustrate an intermittent-catheter system 300 in accordance with some embodiments. Notably, the intermittent-catheter system 300 can have different sealing members joining a distal end piece 164 and a proximal end piece 166 of an intermittent-catheter assembly 310 together as shown among FIGS. 11-13, 14, 15, and 16.

As shown, the intermittent-catheter system 300 includes the intermittent-catheter assembly 310 and, optionally, the drainage bag 120, whether the drainage bag 120 is the rolled drainage bag 118, the bellowed drainage bag 122 or 124, or some other drainage bag such as a folded drainage bag. While not shown, it should be understood that the intermittent-catheter system 300 can include additional catheterization components such as gloves, an antiseptic, an underpad, instructions for use, or some combination thereof.

The intermittent-catheter assembly 310 includes an intermittent catheter 230, a distal end piece 164, the storage sheath 104, and a sealing member 168 joining together the distal end piece 164 and the proximal end piece 166 of the intermittent catheter 230 set forth below. Indeed, the sealing member 168 joins both the distal end piece 164 and the proximal end piece 166 together for a compact form of the intermittent-catheter system 300 or the intermittent-catheter assembly 310 in at least an unused state thereof.

The intermittent catheter 230 includes the catheter tube 106, the plurality of eyelets 108 in the distal end portion of the catheter tube 106, and a proximal end piece 166 akin to the connecting funnel 112 of the intermittent catheter 130 fixedly disposed over the proximal end portion of the catheter tube 106 for draining urine from the intermittent catheter 230. The intermittent catheter 230 can be configured for use by any sex (e.g., male or female) or all sexes (i.e., unisex).

A protection mechanism including a combination of the proximal end piece 166 of the intermittent catheter 230, the distal end piece 164 of the intermittent-catheter assembly 310, the storage sheath 104 of the intermittent-catheter assembly 310 between the proximal end piece 166 and the distal end piece 164, the sealing member 168 over a distal opening of the distal end piece 164 and either over or around a proximal opening of the proximal end piece 166, and, optionally, at least the connecting spout 116 of the drainage bag 120, is configured to enclose an entirety of the catheter tube 106 of the intermittent catheter 230 therein. Notably, the protection mechanism can further include an introducer membrane 172 over the distal opening of the distal end piece 164 but under the sealing member 168. Whether or not the protection mechanism includes the introducer membrane 172 over the distal opening of the distal end piece 164, the protection mechanism is configured to maintain sterility of the catheter tube 106 of the intermittent catheter 230 in an unused state of the intermittent-catheter system 300 or the intermittent-catheter assembly 310 thereof. When lubricated with either a wet or dry lubricant, the protection mechanism is also configured to maintain lubricity of the catheter tube 106 in the unused state of the intermittent-catheter system 300 or the intermittent-catheter assembly 310 thereof. Lastly, when the distal end piece 164 of the intermittent-catheter assembly 310 is configured (e.g., sized and shaped) to ouroborosly insert into the proximal end piece 166 of the intermittent catheter 230, the protection mechanism (sans the sealing member 168) is also configured to prevent residual urine from leaking from the intermittent-catheter assembly 310 in a storage state of the intermittent-catheter system 300 or the intermittent-catheter assembly 310 thereof.

While the proximal end piece 166 of the intermittent catheter 230 is fixedly disposed over the proximal end portion of the catheter tube 106 of the intermittent catheter 230, the distal end piece 164 of the intermittent-catheter assembly 310 is slidably disposed over a distal end portion of the catheter tube 106 in the storage state of the intermittent-catheter system 300 or the intermittent-catheter assembly 310 thereof. However, in an opened state of the intermittent-catheter system 300 or the intermittent-catheter assembly 310 thereof such as when the intermittent-catheter assembly 310 is prepared for catheterization, during the catheterization, or subsequent to the catheterization, the distal end piece 164 is slidably disposed over the medial portion of the catheter tube 106 or the distal end portion of the catheter tube 106 with the storage sheath 104 collapsed between the distal end piece 164 and the proximal end piece 166 of the intermittent catheter 230.

Like that set forth above, the storage sheath 104 of the intermittent-catheter assembly 310 is a thin-walled (e.g., 25-125 μm), relatively small-diameter tube between the proximal end piece 166 and the distal end piece 164 configured to enclose an entirety of the catheter tube 106 of the intermittent catheter 230 therein. Such a storage sheath 104 can be formed of any polymer selected from at least a thermoplastic polyurethane, a thermoplastic elastomer, a polyamide such as a nylon or Pebax®, a thermoplastic polyester such as Hytrel® or Arnitel®, an ethylene vinyl acetate, a polyvinylidene fluoride, a polypropylene, a polyethylene, and the like.

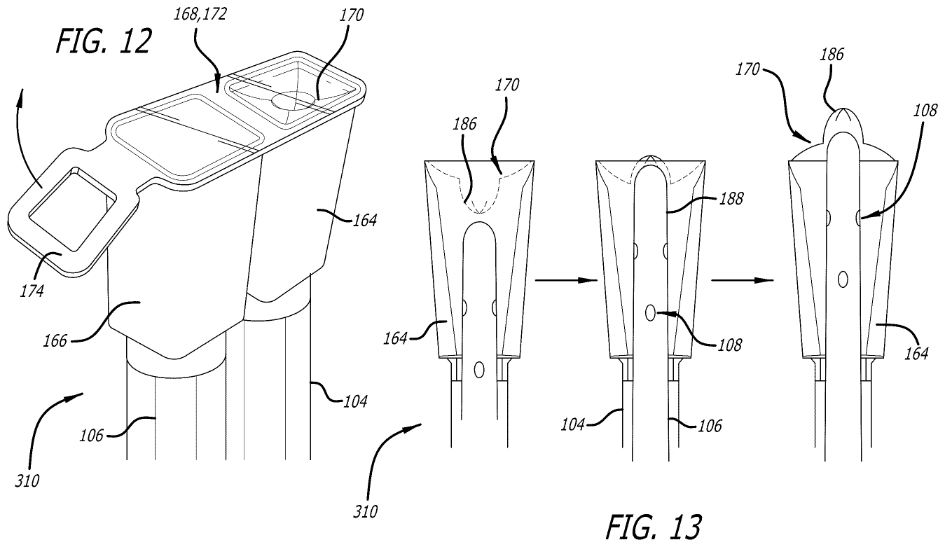

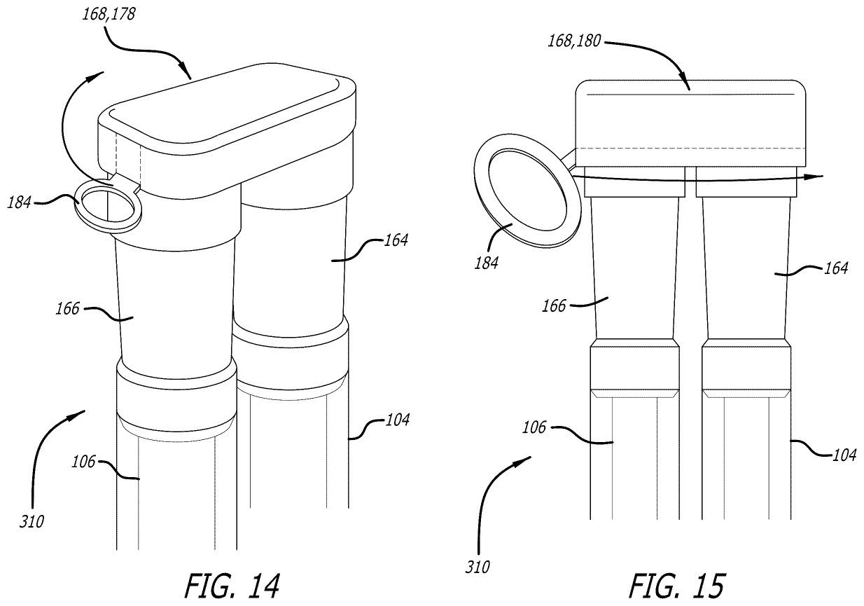

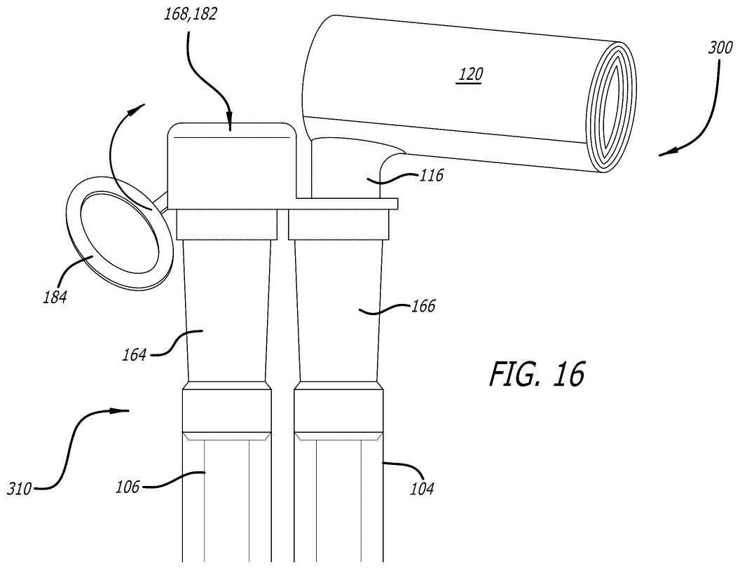

The sealing member 168 is configured to join together the distal end piece 164 and the proximal end piece 166 of the intermittent catheter 230 while simultaneously scaling at least the distal end piece 164 of the intermittent catheter 230. While the sealing member 168 need not seal the proximal end piece 166 of the intermittent catheter 230, it should be understood the proximal end piece 166 of the intermittent catheter 230 is still sealed in the unused state of the intermittent-catheter system 300 or the intermittent-catheter assembly 310 by some other sealing means for sealing the proximal end piece 166 of the intermittent catheter 230 such as by connection of the connecting spout 116 of the drainage bag 120. Such a sealing member 168 can be any cover selected from at least a membrane, a cap, a lid, and a stopper (e.g., a double stopper). In an example, the sealing member 168 can be a sealing membrane 172 as shown in FIGS. 11 and 12. Such a scaling membrane 172 can include an extension tab 174 configured for grasping and peeling the sealing membrane 172 away from the distal opening of the distal end piece 164 of the intermittent-catheter assembly 310 and the proximal opening of the proximal end piece 166 of the intermittent catheter 230. In another example, the sealing member 168 can be a sealing cap 178 or 180 as shown in FIGS. 14-16. Notably, the scaling caps 178 and 180 of FIGS. 14 and 15 extend over both the distal opening of the distal end piece 164 of the intermittent-catheter assembly 310 and the proximal opening of the proximal end piece 166 of the intermittent catheter 230. Differently, the scaling cap 182 of FIG. 16 extends over the distal opening of the distal end piece 164 of the intermittent-catheter assembly 310 and around the proximal opening of the proximal end piece 166 of the intermittent catheter 230 while the connecting spout 116 of the drainage bag 120 is connected to the proximal end piece 166. The sealing cap 178, 180, or 182 can include an extension tab 184 of any size or shape for removing the sealing cap 178, 180, or 182 from the intermittent-catheter assembly 310 thereby. Indeed, as shown in FIG. 14, the extension tab 184 can be a ring configured for grasping and peeling a linear portion of the sealing cap 178 from away from a remainder of the sealing cap 178, thereby disrupting a structural integrity of the sealing cap 178 and loosening the sealing cap 178 for removal from the intermittent-catheter assembly 310. Alternatively, as shown in FIGS. 15 and 16, the extension tab 184 can be a ring configured for grasping and pulling a rip cord through the sealing cap 180 or 182, thereby disrupting the structural integrity of the sealing cap 180 or 182 and loosening the sealing cap 180 or 182 for removal from the intermittent-catheter assembly 310.

The introducer membrane 172 can be formed of a polymer such as an elastomer, wherein the introducer membrane 172 has one or more scores 186 configured to allow a catheter tip 188 of the catheter tube 106 controllably therethrough when the catheter tip 188 is gently pushed through the introducer membrane 172 as shown in FIG. 13. Alternatively, the introducer membrane 172 can be formed of a metal foil, optionally, with a polymeric film thereover, wherein the introducer membrane 172 is configured to rupture when the catheter tip 188 of the catheter tube 106 is pushed through the introducer membrane 172.

Notably, the intermittent-catheter assembly 310 can further include, as set forth above, the one-or-more annular members 114 slidably disposed over the catheter tube 106 of the intermittent catheter 230 but under the storage sheath 104 between the proximal end piece 166 of the intermittent catheter 230 and the distal end piece 164 of the intermittent-catheter assembly 310. The one-or-more annular members 114 are configured as stocky handling points for low-dexterity users when handling the intermittent-catheter assembly 310 during catheterization, particularly, catheter insertion.

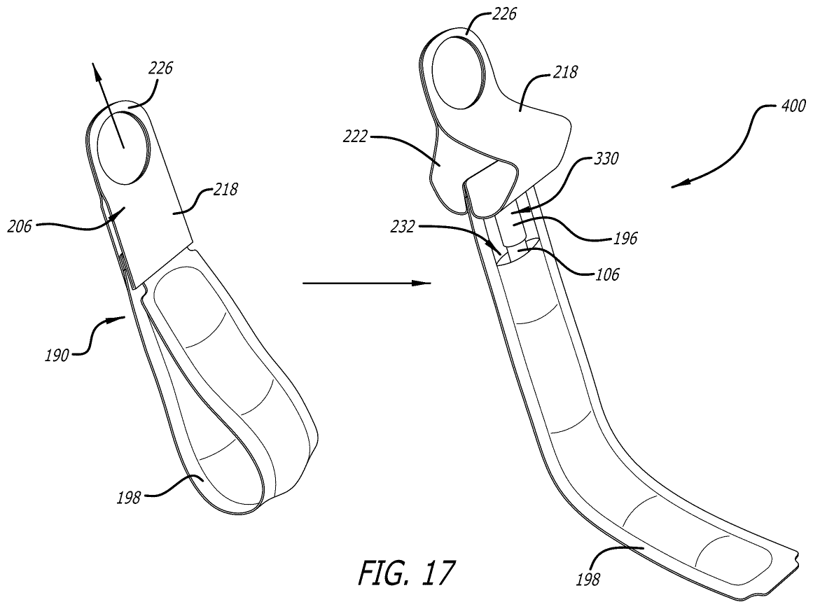

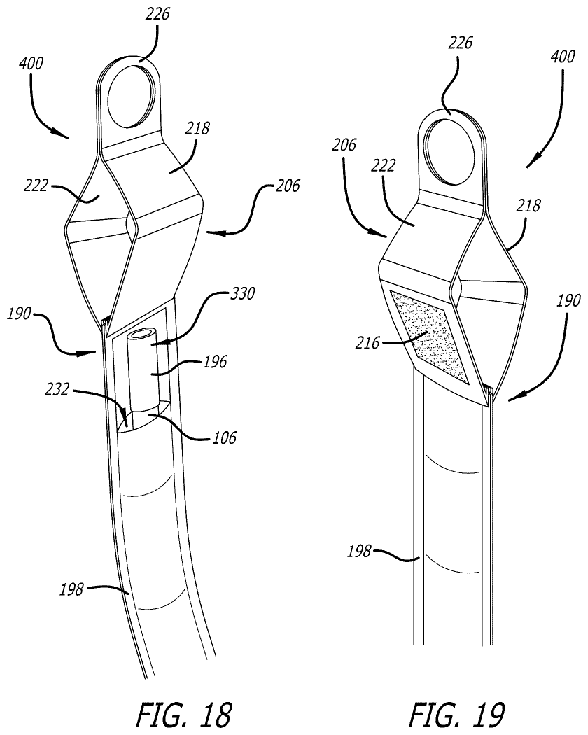

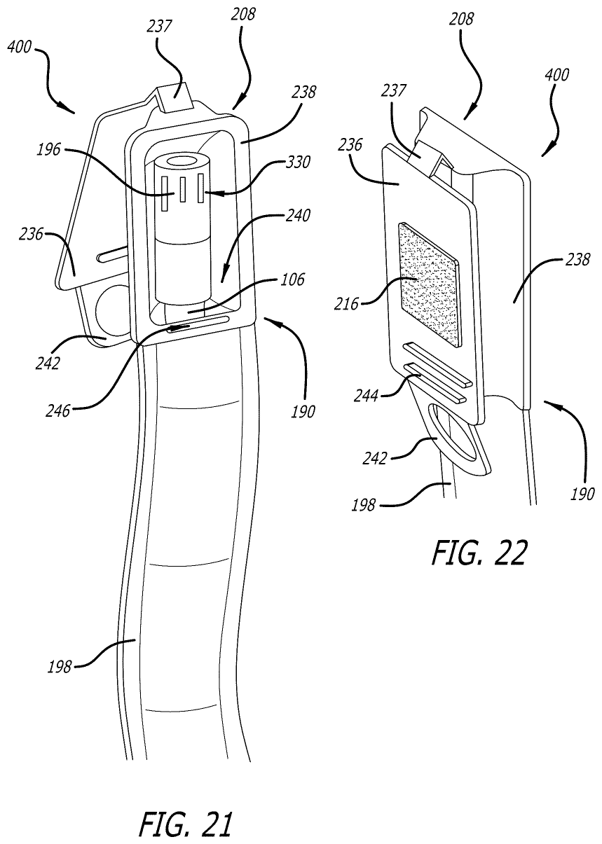

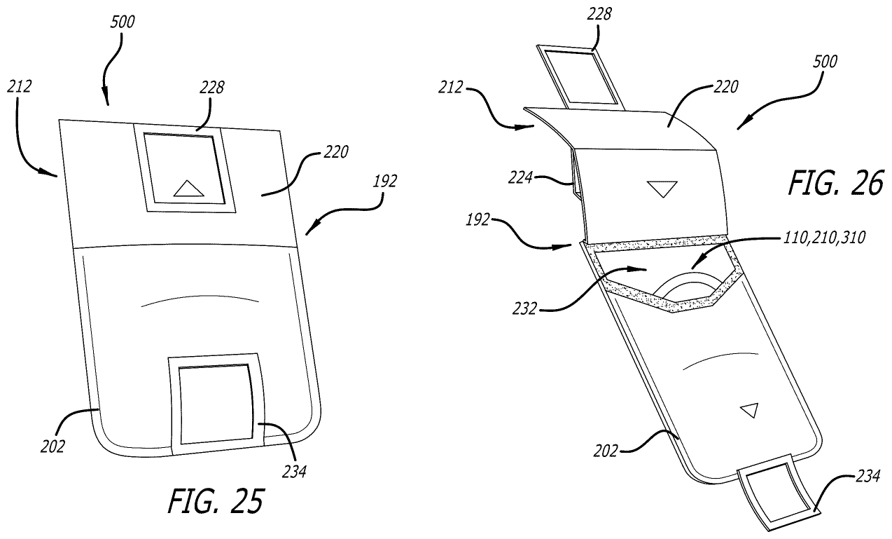

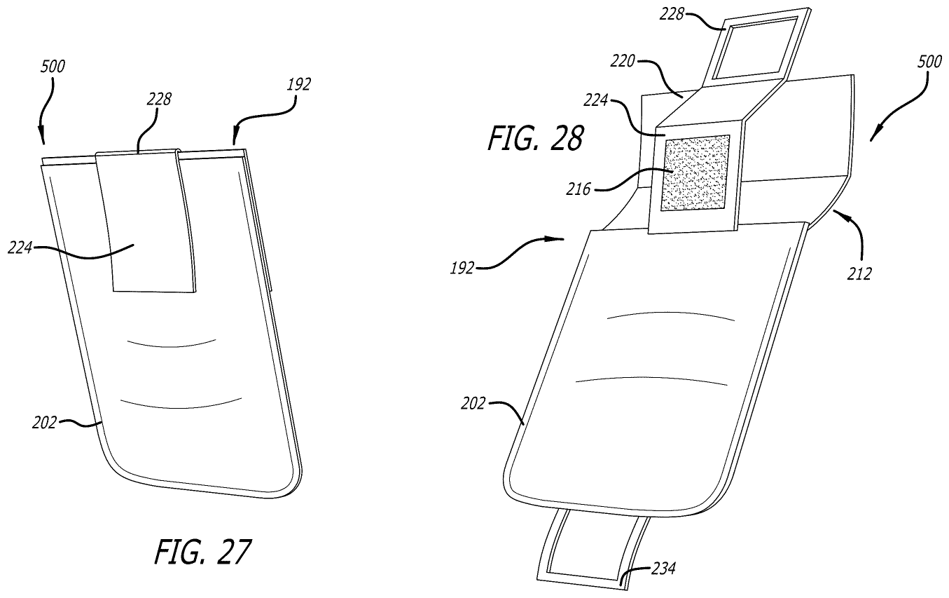

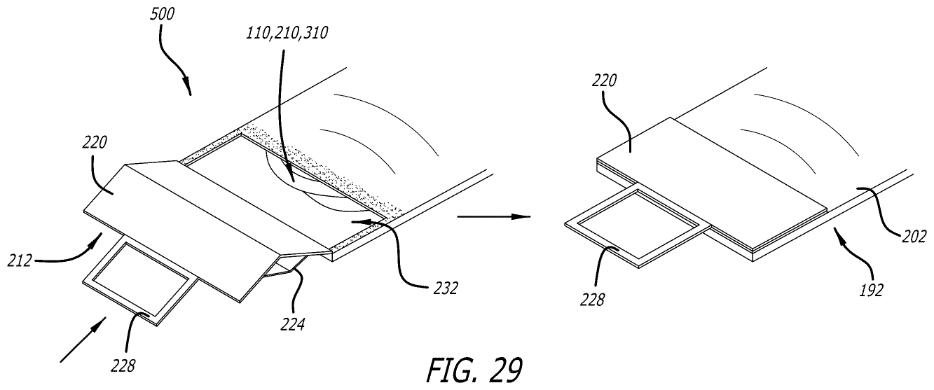

FIGS. 17-22 and 25-32 illustrate intermittent-catheter systems 400, 500, and 600 in accordance with some embodiments. Indeed, FIGS. 17-19 and 20-22 illustrate the intermittent-catheter system 400 including an intermittent catheter 330 in a linear packaging format in accordance with some embodiments. Notably, the packaging 190 for the intermittent catheter 330 in the linear packaging format of the intermittent-catheter system 400 can vary as shown among FIGS. 17-19 and 20-22. In addition, FIGS. 25-29 and 30-32 illustrate the intermittent-catheter systems 500 and 600 including the intermittent catheter 330 in a flat packaging format in accordance with some embodiments. Notably, the packaging 192 and 194 for the intermittent catheter 330 in the flat packaging format of the intermittent-catheter system 500 and 600 can vary as shown among FIGS. 25-29 and 30-32.

As shown, the intermittent-catheter system 400, 500, or 600 includes the intermittent catheter 330 in a linear or flat packaging format defined by packaging 190, 192, or 194. Notably, at least the packaging 194 of the intermittent-catheter system 600 can accommodate the drainage bag 120 in some embodiments, whether the drainage bag 120 is the rolled drainage bag 118, the bellowed drainage bag 122 or 124, or some other drainage bag such as a folded drainage bag. In addition, at least the packaging 194 of the intermittent-catheter system 600 can further accommodate drainage tubing for connecting the intermittent catheter 330 to the drainage bag 120 in some embodiments. While not shown, it should be understood that the intermittent-catheter system 400, 500, or 600 can include additional catheterization components such as gloves, an antiseptic, an underpad, instructions for use, or some combination thereof; however, the additional catheterization components need not be packaged in the packaging 190, 192, or 194 of the intermittent-catheter system 400, 500, or 600.

The intermittent catheter 330 includes the catheter tube 106, the plurality of eyelets 108 in the distal end portion of the catheter tube 106, and a funnel 196 fixedly disposed over the proximal end portion of the catheter tube 106 for draining urine from the intermittent catheter 330. The intermittent catheter 330 can be configured for use by any sex (e.g., male or female) or all sexes (i.e., unisex).

The packaging 190, 192, or 194 of the intermittent-catheter system 400, 500, or 600 encloses at least the intermittent catheter 330 therein to maintain sterility of the intermittent catheter 330 in at least an unused state of the intermittent-catheter system 400, 500, or 600. Such packaging 190, 192, or 194 includes at least a flexible packaging body 198, 202, or 204 and a flexible or rigid packaging cover 206, 208, 212, or 214. The packaging body 198, 202, or 204 is a perimetrically sealed pouch or the like configured to hold at least the catheter tube 106 of the intermittent catheter 330 therein. The packaging cover 206, 208, 212, or 214 is configured to reveal the funnel 196 of the intermittent catheter 330 when an uncovering operation is performed with the packaging cover 206, 208, 212, or 214. Such an uncovering operation advantageously reveals a securing mechanism for securing the intermittent-catheter system 400, 500, or 600 for catheterization. Indeed, such a securing mechanism can include an adhesive patch 216 on either the packaging body 198, 202, or 204 or the packaging cover 198, 202, or 204 for adhering the packaging 190, 192, or 194 to a vertical surface (e.g., a wall, a partition, a door, a mirror, etc.) for removal of the intermittent catheter 330 from the packaging 190, 192, or 194 for the catheterization.

Adverting to the intermittent-catheter systems 400 and 500 and some particular features thereof, FIGS. 17-19 and 25-29 illustrate the intermittent-catheter system 400 and 500 with the packaging covers 206 and 212 as flexible pull covers in accordance with some embodiments.

As shown, the packaging cover 206 or 212 is flexible with an anterior cover 218 or 220 and a posterior cover 222 or 224. The anterior cover 218 or 220 and the posterior cover 222 or 224 are coupled together through an extension tab 226 or 228 at a tabbed end portion of the anterior cover 218 or 220 and a tabbed end portion of the posterior cover 222 or 224. Notably, the extension tab 226 is formed of the tabbed end portions of the anterior and posterior covers 218 and 222 sealed together, whereas the extension tab 228 is a separate piece coupled to the tabbed end portions of the anterior and posterior covers 220 and 224. The anterior cover 218 or 220 and the posterior cover 222 or 224 are also coupled together through an access end portion of the packaging body 198 or 202 at a coupled end portion of the anterior cover 218 or 220, which is opposite the tabbed end portion of the anterior cover 218 or 220, and a coupled end portion of the posterior cover 222 or 224, which is likewise opposite the tabbed end portion of the posterior cover 222 or 224. The coupled end portion of the anterior cover 218 or 220, the access end portion of the packaging body 198 or 202, and the coupled end portion of the posterior cover 222 or 224 are sealed together akin to the extension tab 226.

Further to the packaging cover 206 or 212, the anterior cover 218 or 220 is folded over an opening 232 in an anterior face of the packaging body 198 or 202, and the posterior cover 222 or 224 is folded over a posterior face of the packaging body 198 or 202 in at least the unused state of the intermittent-catheter system 400 or 500. Indeed, an internal face of the anterior cover 218 or 220 is adhered to the packaging body 198 or 202 by an adhesive around a perimeter of the internal face of the anterior cover 218 or 220, the opening 232 in the anterior face of the packaging body 198 or 202, or both, which adhesive is notably configured for rescaling the intermittent catheter 330 in the packaging 190 or 192 subsequent to catheterization for a storage state of the intermittent-catheter system 400 and 500. Relatedly, an internal face of the posterior cover 222 or 224 is adhered to the packaging body 198 or 202 by the adhesive patch 216 in the unused state of the intermittent-catheter system 400 or 500, which adhesive patch 216 is on either the internal face of the posterior cover 222 or 224, as shown, or the posterior face of the packaging body 198 or 202. Notably, while the adhesive patch 216 might be on the internal face of the posterior cover 222 or 224 and adhered to the packaging body 198 or 202 prior to the uncovering operation, the adhesive patch 216 is on an external face of the posterior cover 222 or 224 subsequent to the uncovering operation for adhering the packaging 190 or 192 to the foregoing vertical surface for removal of the intermittent catheter 330 from the packaging 190 or 192.

As to the uncovering operation, the anterior cover 218 or 220 and the posterior cover 222 or 224 of the packaging cover 206 or 212 are configured to simultaneously peel away from the packaging body 198 or 202 when the extension tab 226 or 228 is pulled away from an end portion of the packaging body 198 or 202, whether the end portion of the packaging body 198 or 202 is the access end portion of the packaging body 198 to which the anterior cover 218 or 220 and the posterior cover 222 or 224 are coupled or a closed end of the packaging body 198 or 202 opposite the access end portion of the packaging body 198. Notably, the packaging body 198 or 202 can include a complementary extension tab 234 to the extension tab 226 or 228 of the packaging cover 206 or 212. When present, such an extension tab 234 is coupled to the closed end portion of the packaging body 198 or 202 for grasping while pulling the extension tab 226 or 228 of the packaging cover 206 or 212 away from at least the access end portion of the packaging body 198 or 202 in the uncovering operation.

Continuing with the uncovering operation for the packaging 190 of the intermittent-catheter system 400, when the internal face of the anterior cover 218 is adhered to the packaging body 198 by the adhesive around the perimeter of the internal face of the anterior cover 218, the closed end portion of the packaging body 198 can also be adhered to the internal face of the anterior cover 218. As shown in FIG. 17, the packaging 190 and the intermittent catheter 330 disposed therein are thusly folded into a compact form in at least the unused state of the intermittent-catheter system 400. The closed end portion of the packaging body 198 is released by the anterior cover 218 when the extension tab 226 of the packaging cover 206 is pulled away from the access end portion of the packaging body 198 in the uncovering operation, thereby unfolding the intermittent-catheter system 400, opening the packaging 190 for access to the intermittent catheter 330, and revealing the adhesive patch 216 for adhering the packaging 190 to the foregoing vertical surface in a single operation.

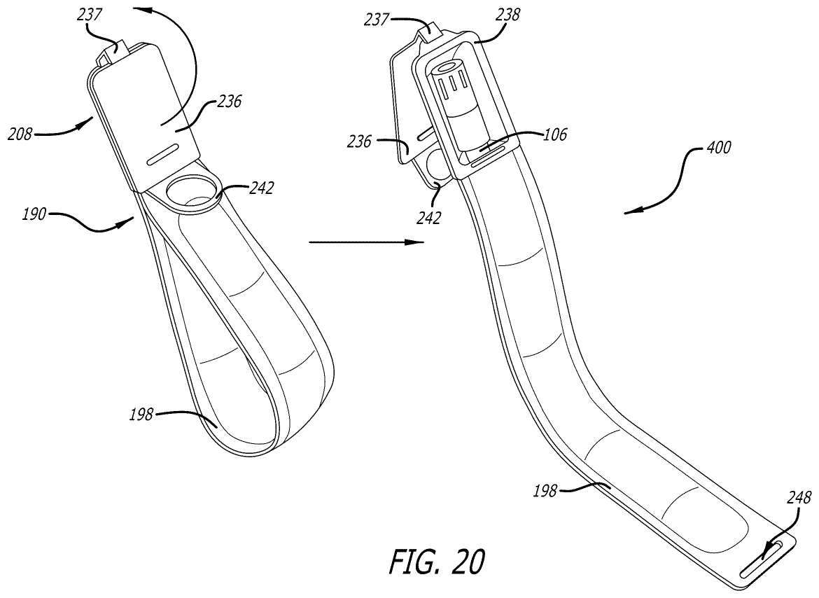

Adverting to the intermittent-catheter system 400 and some other particular features thereof, FIGS. 20-22 illustrate the intermittent-catheter system 400 with the packaging cover 208 as a rigid cover in accordance with some embodiments.

As shown, the packaging cover 208 is rigid with an anterior cover 236 and a posterior tray 238 configured to hold the funnel 196 of the intermittent catheter 330. The anterior cover 236 and the posterior tray 238 are coupled together through a hinge (e.g., a living hinge) at a hinged end portion of the anterior cover 236 and a hinged end portion of the posterior tray 238. The posterior tray 238 is also coupled to the access end portion of the packaging body 198 at a coupled end portion of the posterior tray 238, which is opposite the hinged end portion of the posterior tray 238.

Further to the packaging cover 208, the anterior cover 236 is snapped onto the posterior tray 238 over an opening 240 in the posterior tray 238 in at least the unused state of the intermittent-catheter system 400. The anterior cover 236 is configured to swing away from the opening 240 in the posterior tray 238 in accordance with the hinge when an extension tab 242 extending from the anterior cover 236 is pulled away from the access end portion of the packaging body 198 in the uncovering operation. Not unlike some embodiments of the posterior cover 222 or 224 of the packaging cover 206 or 212, an internal face of the anterior cover 236 includes the adhesive patch 216 advantageously revealed by way of the uncovering operation for adhering the packaging 190 to the foregoing vertical surface for removal of the intermittent catheter 330 from the packaging 190 for catheterization. Notably, while the adhesive patch 216 might be on the internal face of the anterior cover 236 prior to the uncovering operation, the adhesive patch 216 is on an external face of the anterior cover 236 subsequent to the uncovering operation for adhering the packaging 190 to the foregoing vertical surface for removal of the intermittent catheter 330 from the packaging 190.

Continuing with the uncovering operation for the packaging 190 of the intermittent-catheter system 400, when the anterior cover 236 is snapped onto the posterior tray 238 over the opening 240 in the posterior tray 238, a closed end of the packaging body 198 opposite the access end portion of the packaging body 198 can also held between the anterior cover 236 and the posterior tray 238. For example, the anterior cover 236 and the posterior tray 238 can have interlocking features such as an elongate protrusion 244 and complementary channel or groove 246 configured to hold the closed end of the packaging body 198 therebetween by way of an elongate through hole 248 through the closed end of the packaging body 198. As shown in FIG. 20, the packaging 190 and the intermittent catheter 330 disposed therein are thusly folded into a compact form in at least the unused state of the intermittent-catheter system 400. The closed end portion of the packaging body 198 is released by the anterior cover 236 when the extension tab 242 is pulled away from the access end portion of the packaging body 198 in the uncovering operation, thereby unfolding the intermittent-catheter system 400, opening the packaging 190 for access to the intermittent catheter 330, and revealing the adhesive patch 216 for adhering the packaging 190 to the foregoing vertical surface in a single operation.

Notably, the anterior cover 236 is also configured to swing back toward the opening 240 in the posterior tray 238 in accordance with the hinge when the extension tab 242 extending from the anterior cover 236 is pulled back toward the access end portion of the packaging body 198. Indeed, the packaging cover 208 is configured for resealing the intermittent catheter 330 in the packaging 190 subsequent to catheterization by snapping the anterior cover 236 back onto the posterior tray 238 over the opening 240 in the posterior tray 238 for the storage state of the intermittent-catheter system 400.

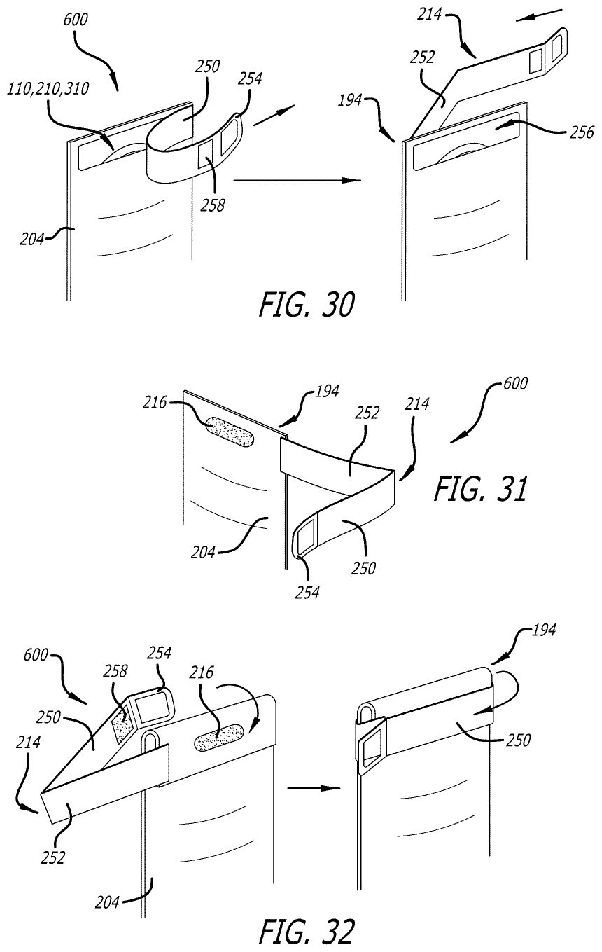

Adverting to the intermittent-catheter system 600 and some particular features thereof, FIGS. 30-32 illustrate the intermittent-catheter system 600 with the packaging cover 214 as a flexible wraparound cover in accordance with some embodiments.

As shown, the packaging cover 214 is flexible with an anterior cover 250 and a posterior cover 252. The anterior cover 250 and the posterior cover 252 are coupled together by their longitudinal end portions in a middle portion of the packaging cover 214, thereby forming the packaging cover 214 in a unitary piece in at least some embodiments. The packaging cover 214 is coupled to a posterior face of the packaging body 204 in an access end portion thereof through a coupled end portion of the packaging cover 214, which coupled end portion of the packaging cover 214 is opposite a tabbed end portion of the packaging cover 214 over an anterior face of the packaging body 204 in the access end portion thereof. Notably, the tabbed end portion of the packaging cover 214 includes an extension tab 254 or the like.

Further to the packaging cover 214, the anterior cover 250 is over an opening 256 in the anterior face of the packaging body 204 while the posterior cover 252 is over the posterior face of the packaging body 204 in at least the unused state of the intermittent-catheter system 600. Indeed, an internal face of the anterior cover 250 is adhered to the packaging body 204 by an adhesive around at least a perimeter of the internal face of the anterior cover 250, the opening 256 in the anterior face of the packaging body 204, or both, which adhesive is notably configured for resealing the intermittent catheter 330 in the packaging 194 subsequent to catheterization for a storage state of the intermittent-catheter system 600. Relatedly, an internal face of the posterior cover 252 is adhered to the packaging body 204 by the adhesive patch 216 prior to the uncovering operation such as in the unused state of the intermittent-catheter system 600, which adhesive patch 216 is on either the posterior face of the packaging body 204, as shown, or the internal face of the posterior cover 252. Whether the adhesive patch 216 is on the posterior face of the packaging body 204 or the internal face of the posterior cover 252, the adhesive patch 216 is configured for adhering the packaging 194 to the foregoing vertical surface subsequent to the uncovering operation for removal of the intermittent catheter 330 from the packaging 194.

As to the uncovering operation, the anterior cover 250 and the posterior cover 252 of the packaging cover 214 are configured to sequentially peel away from the packaging body 204 when the packaging cover 214 is pulled across the access end portion of the packaging body 204 by the extension tab 254 extending from the anterior cover 250. Indeed, the anterior cover 250 initially peels away from the opening 256 in the anterior face of the packaging body 204, and the posterior cover 252 subsequently peels away from the posterior face of the packaging body 204 during the uncovering operation. Again, the adhesive patch 216 is on the posterior face of the packaging body 204 or the internal face of the posterior cover 252. As such, upon pulling the packaging cover 214 across the access end portion of the packaging body 204 by the extension tab 254 in the uncovering operation, the packaging 194 is opened for access to the intermittent catheter 330, and the adhesive patch 216 is also revealed for adhering the packaging 194 to the foregoing vertical surface in a single operation.

Notably, the access end portion of the packaging body 204 is configured to fold over a main portion of the packaging body 204 as shown in FIG. 32 for rescaling the intermittent catheter 330 in the packaging 194 with any adhesive around the opening 256 in the anterior face of the packaging body 204 for the storage state of the intermittent-catheter system 600. Further, the adhesive patch 216 on the posterior face of the packaging body 204 or an additional adhesive patch 258 adjacent to the extension tab 254 in the tabbed end portion of the packaging cover 214 is configured for securing the access end portion of the packaging body 204 to the main portion of the packaging body 204 with the packaging cover 214 when wrapped back around the packaging body 204 in the storage state of the intermittent-catheter system 600.

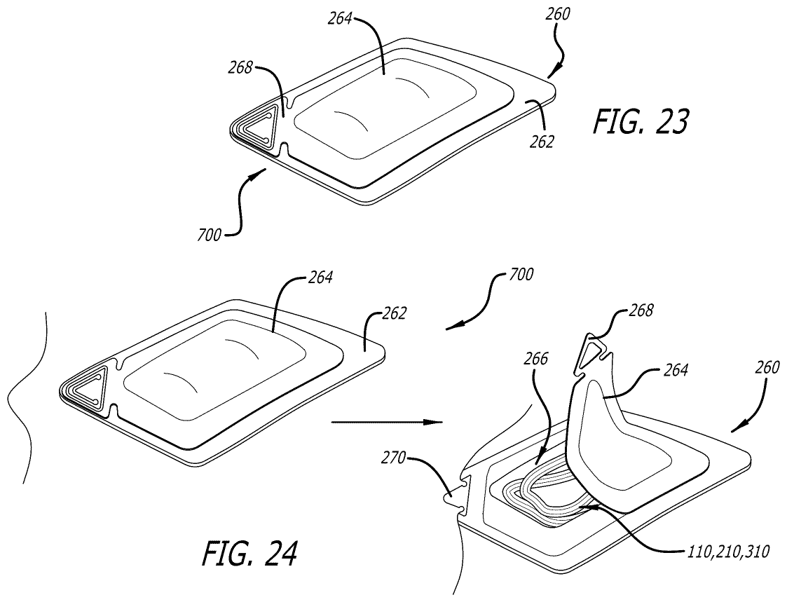

FIGS. 23 and 24 illustrate an intermittent-catheter system 700 in accordance with some embodiments. Indeed, FIGS. 23 and 24 illustrate the intermittent-catheter system 700 including the intermittent-catheter assembly 110, 210, or 310 in another flat packaging format in accordance with some embodiments. However, it should be understood that the intermittent-catheter system 700 is not limited to the intermittent-catheter assembly 110, 210, or 310, as set forth below.

As shown, the intermittent-catheter system 700 includes the intermittent-catheter assembly 110, 210, or 310 in a flat packaging format defined by packaging 260. Notably, the packaging 260 can be configured to accommodate the intermittent-catheter assembly 110 or 210 and the drainage bag 120 included therewith, the intermittent-catheter assembly 310 and the drainage bag 120 in addition thereto, or the intermittent-catheter assembly 310 without the drainage bag 120. When present, the drainage bag 120 is the rolled drainage bag 118, the bellowed drainage bag 122 or 124, or some other drainage bag such as a folded drainage bag. In addition, the packaging 260 of the intermittent-catheter system 700 can be configured to accommodate drainage tubing for connecting the intermittent catheter 230 of the intermittent-catheter assembly 310 to the drainage bag 120 in some embodiments. While not shown, it should be understood that the intermittent-catheter system 700 can include additional catheterization components such as gloves, an antiseptic, an underpad, instructions for use, or some combination thereof. While the additional catheterization components can be packaged in the packaging 260, the additional catheterization components need not be packaged in the packaging 260 of the intermittent-catheter system 700.

The packaging 260 of the intermittent-catheter system 700 encloses at least the intermittent-catheter assembly 110, 210, or 310 therein to maintain sterility of the intermittent-catheter assembly 110, 210, or 310 in at least an unused state of the intermittent-catheter system 700. Such packaging 260 includes at least a flexible packaging body 262 and a flexible packaging cover 264 over an opening 266 in an anterior face of the packaging body 262, thereby forming a face-opening pouch. Indeed, the packaging cover 264 is sealed by an adhesive around an outer perimeter of an internal face of the packaging cover 264, an inner perimeter of the anterior face of the packaging body 262, or both, such that at least the intermittent-catheter assembly 110, 210, or 310 is revealed within the opening 266 in the anterior face of the packaging body 262 when an uncovering operation is performed with the packaging cover 264.

As to the uncovering operation, the packaging cover 264 includes a start tab 268 unattached to the packaging body 262 in an edge or corner of the packaging 260, which start tab 268 is configured for grasping and peeling the packaging cover 264 away from the packaging body 262 to reveal the intermittent-catheter assembly 110, 210, or 310 within the opening 266 in the anterior face of the packaging body 262. Advantageously, the uncovering operation also reveals a securing mechanism under the start tab 268 of the packaging cover 264 for securing the intermittent-catheter system 700 for catheterization. The securing mechanism can include a rigid clip 270 integrated into the edge or corner of the packaging body 262, wherein the clip 270 is configured for clipping the packaging 260 of the intermittent-catheter system 700 to a sheet of flexible material (e.g., shirt, bedsheet, etc.) for removal of the intermittent-catheter assembly 110, 210, or 310 from the packaging 260 for the catheterization. After the catheterization, with at least the intermittent-catheter assembly 110, 210, or 310 placed back into the packaging body 262, the adhesive around the packaging cover 264 or the packaging body 262 can be reused as configured to reseal the intermittent-catheter assembly 110, 210, or 310 in the packaging 260 for a storage state of the intermittent-catheter system 700.

Notably, flexible packaging such as that of the packaging 260 offers better portability than rigid packaging in that the intermittent-catheter system 700 or the like can be easily and tightly packed in luggage; however, such flexible packaging can be less protective than rigid packaging when the intermittent-catheter system 700 or the like is independently carried or otherwise toted.

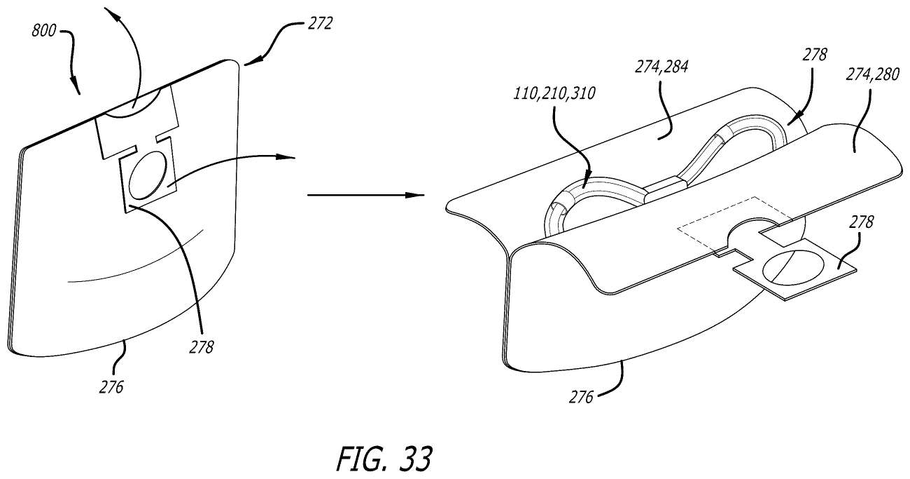

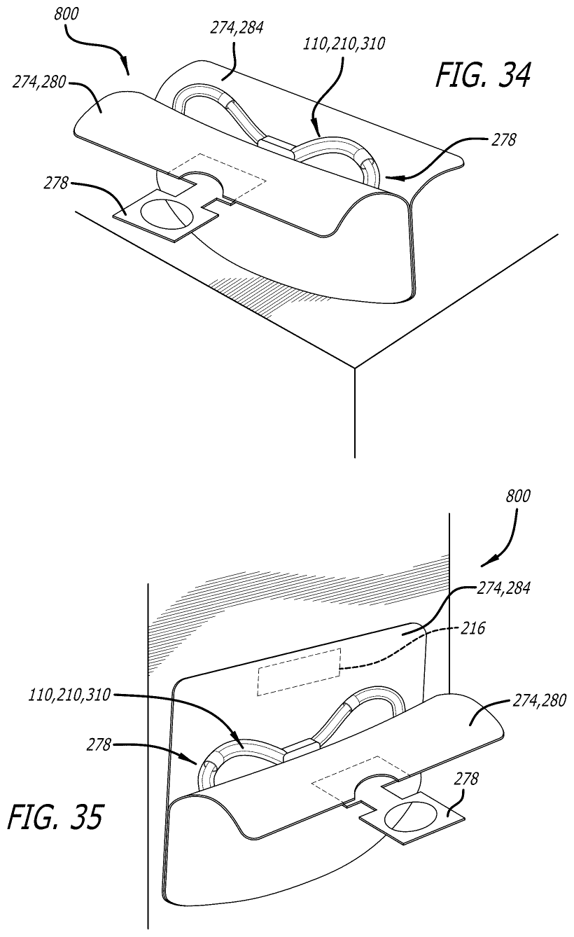

FIGS. 33-35 illustrate an intermittent-catheter system 800 in accordance with some embodiments. Indeed, FIGS. 33-35 illustrate the intermittent-catheter system 800 including the intermittent-catheter assembly 110, 210, or 310 in another flat packaging format in accordance with some embodiments. However, it should be understood that the intermittent-catheter system 800 is not limited to the intermittent-catheter assembly 110, 210, or 310, as set forth below.

As shown, the intermittent-catheter system 800, like the intermittent-catheter system 700, includes the intermittent-catheter assembly 110, 210, or 310 in a flat packaging format defined by packaging 272; however, different than the intermittent-catheter system 700, the flat packaging format defined by the packaging 272 is configured for use in a vertical orientation instead of a horizontal orientation like that defined by the packaging 260. The packaging 272 can be configured to accommodate the intermittent-catheter assembly 110 or 210 and the drainage bag 120 included therewith, the intermittent-catheter assembly 310 and the drainage bag 120 in addition thereto, or the intermittent-catheter assembly 310 without the drainage bag 120. When present, the drainage bag 120 is the rolled drainage bag 118, the bellowed drainage bag 122 or 124, or some other drainage bag such as a folded drainage bag. In addition, the packaging 272 of the intermittent-catheter system 800 can be configured to accommodate drainage tubing for connecting the intermittent catheter 230 of the intermittent-catheter assembly 310 to the drainage bag 120 in some embodiments. While not shown, it should be understood that the intermittent-catheter system 800 can include additional catheterization components such as gloves, an antiseptic, an underpad, instructions for use, or some combination thereof. While the additional catheterization components can be packaged in the packaging 272, the additional catheterization components need not be packaged in the packaging 272 of the intermittent-catheter system 800.

The packaging 272 of the intermittent-catheter system 800 encloses at least the intermittent-catheter assembly 110, 210, or 310 therein to maintain sterility of the intermittent-catheter assembly 110, 210, or 310 in at least an unused state of the intermittent-catheter system 800. Such packaging 272 includes at least flexible sides 274 and a flexible or rigid bottom 276, wherein the sides 274 of the packaging 272 are sealed together at their margins as well as around the bottom 276 of the packaging 272. Opposite the bottom 276 of the packaging 272 is a top of the packaging 272 including an opening 278, which opening 278 is sealed by the sides 274 of the packaging 272 in the unused state of the intermittent-catheter system 800 for a top-opening pouch. Indeed, the margins of the sides 274 of the packaging 272 are sealed by an adhesive such that at least the intermittent-catheter assembly 110, 210, or 310 is revealed within the opening 278 in the top of the packaging 272 when an opening operation is performed with the sides 274 of the packaging 272.

As to the opening operation, an anterior side 280 of the sides 274 of the packaging 272 includes an extension tab 282 extending therefrom in the top of the packaging 272, which extension tab 282 is configured for grasping and peeling the anterior side 280 away from a posterior side 284 of the sides 274 of the packaging 272 to reveal the intermittent-catheter assembly 110, 210, or 310 within the opening 278 formed in the top of the packaging 272. Notably, the posterior side 284 of the packaging 272 can include an extension tab like the extension tab 282 that extends from the posterior side 284 of the packaging 272; however, as shown, the anterior side 280 of the packaging 272 can alternatively be cut away to reveal a portion of the posterior side 284 of the packaging 272 therebehind, which portion of the posterior side 284 of the packaging 272 can likewise be grasped for peeling the anterior side 280 of the packaging 272 away from the posterior side 284 of the packaging 272 during the opening operation.

Advantageously, the bottom 276 of the packaging 272 is flat for setting the intermittent-catheter system 800 onto a horizontal surface (e.g., a counter, a ledge of a freestanding sink, a lid of a toilet tank, etc.) for removal of the intermittent-catheter assembly 110, 210, or 310 from the packaging 272 for catheterization. Additionally, the posterior side 284 of the packaging 272 can include the adhesive patch 216 for adhering the packaging 272 to a vertical surface (e.g., a wall, a partition, a door, a mirror, etc.) for removal of the intermittent-catheter assembly 110, 210, or 310 from the packaging 272 for the catheterization. After the catheterization, with at least the intermittent-catheter assembly 110, 210, or 310 placed back into the packaging 272, the adhesive around the margins of the sides 274 of the packaging 272 can be reused as configured to reseal the intermittent-catheter assembly 110, 210, or 310 in the packaging 272 for a storage state of the intermittent-catheter system 800.

Notably, flexible packaging such as that of the packaging 272 offers better portability than rigid packaging in that the intermittent-catheter system 800 or the like can be easily and tightly packed in luggage; however, such flexible packaging can be less protective than rigid packaging when the intermittent-catheter system 800 or the like is independently carried or otherwise toted.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

1. An intermittent-catheter system, comprising:
an intermittent-catheter assembly including:
an intermittent catheter including a catheter tube and a connecting funnel over a proximal end portion of the catheter tube for draining urine from the intermittent catheter;
a distal end piece slidably disposed over a distal end portion of the catheter tube; and
a storage sheath between the connecting funnel and the distal end piece, a combination of the connecting funnel, the distal end piece, and the storage sheath configured to enclose an entirety of the catheter tube therein; and
a drainage bag including a connecting spout connected to the connecting funnel of the intermittent catheter in at least an unused state of the intermittent-catheter system.

2. The intermittent-catheter system of claim 1, wherein the connecting funnel of the intermittent catheter is connected to the connecting spout of the drainage bag by a hinge.

3. The intermittent-catheter system of claim 2, wherein the connecting funnel of the intermittent catheter includes a male fitting extending from the connecting funnel, and the connecting spout of the drainage bag includes a female fitting within the connecting spout configured to receive the male fitting.

4. The intermittent-catheter system of claim 2, wherein the connecting funnel of the intermittent catheter is configured to drain the urine into the drainage bag through the connecting spout of the drainage bag when the hinge is in a closed state.

5. The intermittent-catheter system of claim 2, wherein the connecting spout of the drainage bag is configured to drain the urine from the drainage bag when the hinge is in an opened state.

6. The intermittent-catheter system of claim 1, wherein the distal end piece of the intermittent catheter includes a protective seal over a distal opening of the distal end piece in at least the unused state of the intermittent-catheter system, the protective seal configured to maintain sterility of the catheter tube of the intermittent catheter within the combination of the connecting funnel, the distal end piece, and the storage sheath.

7. The intermittent-catheter system of claim 1, wherein the connecting funnel of the intermittent catheter is connected to the connecting spout of the drainage bag by a slip or lock fit.

8. The intermittent-catheter system of claim 7, wherein the connecting spout of the drainage bag includes a male fitting extending from the connecting spout, and the connecting funnel of the intermittent catheter includes a female fitting within the connecting funnel configured to receive the male fitting.

9. The intermittent-catheter system of claim 8, wherein the connecting funnel of the intermittent catheter includes a fluid seal disposed within the female fitting of the connecting funnel, the fluid seal configured to accept the male fitting of the connecting spout therethrough for draining the urine into the drainage bag through the connecting spout when the connecting funnel is connected to the connecting spout.

10. The intermittent-catheter system of claim 9, wherein the fluid seal is configured to close when the male fitting of the connecting spout is withdrawn from the female fitting of the connecting funnel, thereby preventing residual urine in the intermittent catheter from leaking from the intermittent catheter when the connecting funnel is disconnected from the connecting spout.

11. The intermittent-catheter system of claim 8, wherein the connecting spout of the drainage bag is configured to drain the urine from the drainage bag when the connecting funnel of the intermittent catheter is disconnected from the connecting spout.

12. The intermittent-catheter system of claim 11, wherein the male fitting of the connecting spout is configured to drain the urine from the drainage bag.

13. The intermittent-catheter system of claim 11, wherein the connecting spout of the drainage bag includes a side arm at an angle to the male fitting, the side arm configured to drain the urine from the drainage bag.

14. The intermittent-catheter system of claim 1, wherein the connecting funnel is connected to the distal end piece of the intermittent-catheter assembly in an end-to-end connection through a first arm of the connecting funnel, and the connecting funnel of the intermittent catheter is also connected to the connecting spout of the drainage bag through a second arm of the connecting funnel.

15. The intermittent-catheter system of claim 14, wherein the distal end piece of the intermittent-catheter assembly includes a first male fitting extending from the distal end piece, and the first arm of the connecting funnel includes a first female fitting within the first arm configured to receive the first male fitting.

16. The intermittent-catheter system of claim 15, wherein the first arm of the connecting funnel includes a first fluid seal disposed within the first female fitting of the first arm, the first fluid seal configured to accept the first male fitting of the distal end piece therethrough.

17. The intermittent-catheter system of claim 16, wherein the end-to-end connection between the first arm of the connecting funnel and the distal end piece of the intermittent-catheter assembly is configured to maintain sterility of the catheter tube of the intermittent catheter within the combination of the connecting funnel, the distal end piece, and the storage sheath in at least the unused state of the intermittent-catheter system.

18. The intermittent-catheter system of claim 16, wherein the end-to-end connection between the first arm of the connecting funnel and the distal end piece of the intermittent-catheter assembly is configured to prevent residual urine in the intermittent catheter from leaking from the intermittent catheter in a used state of the intermittent-catheter system.

19. The intermittent-catheter system of claim 16, wherein the first fluid seal disposed within the first female fitting of the first arm is configured to close when the first male fitting of the distal end piece is withdrawn from the first female fitting, thereby preventing the urine in the intermittent catheter from leaking from the first arm of the intermittent catheter while draining the urine into the drainage bag.

20. The intermittent-catheter system of claim 16, wherein the connecting spout of the drainage bag includes a second male fitting extending from the connecting spout, and the second arm of the connecting funnel includes a second female fitting within the second arm configured to receive the second male fitting.

21. The intermittent-catheter system of claim 20, wherein the second arm of the connecting funnel includes a second fluid seal disposed within the second female fitting of the second arm, the second fluid seal configured to accept the second male fitting of the connecting spout therethrough for draining the urine into the drainage bag through the connecting spout when the second arm of the connecting funnel is connected to the connecting spout.

22. The intermittent-catheter system of claim 21, wherein the second fluid seal disposed within the second female fitting of the second arm is configured to close when the second male fitting of the connecting spout is withdrawn from the second female fitting, thereby preventing residual urine in the intermittent catheter from leaking from the intermittent catheter when the connecting funnel is disconnected from the connecting spout.

23. The intermittent-catheter system of claim 1, wherein the drainage bag is in a compact rolled form in at least the unused state of the intermittent-catheter system.

24. The intermittent-catheter system of claim 1, wherein the drainage bag is in a compact bellowed form in at least the unused state of the intermittent-catheter system.

25-54. (canceled)
